SEQUENCE LISTING (107 sequences)

SEQ ID NO: 1 - DNA, 20 bp, synthetic construct
  source 1..20: mol_type = other DNA
tgctcgggattttaccagtt

SEQ ID NO: 2 - DNA, 20 bp, synthetic construct
  source 1..20: mol_type = other DNA
tggctgctcgggattttacc

SEQ ID NO: 3 - DNA, 20 bp, synthetic construct
  source 1..20: mol_type = other DNA
gatgactttggctgctcggg

SEQ ID NO: 4 - DNA, 20 bp, synthetic construct
  source 1..20: mol_type = other DNA
tttctgtggggtgaggatgt

SEQ ID NO: 5 - DNA, 20 bp, synthetic construct
  source 1..20: mol_type = other DNA
agcagtttctgtggggtgag

SEQ ID NO: 6 - DNA, 20 bp, synthetic construct
  source 1..20: mol_type = other DNA
ttcttcagtgtgtccagcag

SEQ ID NO: 7 - DNA, 20 bp, synthetic construct
  source 1..20: mol_type = other DNA
tggaatgaagatagcagatt

SEQ ID NO: 8 - DNA, 20 bp, synthetic construct
  source 1..20: mol_type = other DNA
tagggactggaatgaagata

SEQ ID NO: 9 - DNA, 20 bp, synthetic construct
  source 1..20: mol_type = other DNA
aggggtagggactggaatga

SEQ ID NO: 10 - DNA, 20 bp, synthetic construct
  source 1..20: mol_type = other DNA
gagcaggcactgaggggtag

SEQ ID NO: 11 - DNA, 20 bp, synthetic construct
  source 1..20: mol_type = other DNA
caggttatttggatgagagc

SEQ ID NO: 12 - DNA, 20 bp, synthetic construct
  source 1..20: mol_type = other DNA
gtcacctcccaggttatttg

SEQ ID NO: 13 - DNA, 20 bp, synthetic construct
  source 1..20: mol_type = other DNA
tcctgattgtcacctcccag

SEQ ID NO: 14 - DNA, 20 bp, synthetic construct
  source 1..20: mol_type = other DNA
gcagcagcacaaatggtgtg

SEQ ID NO: 15 - DNA, 20 bp, synthetic construct
  source 1..20: mol_type = other DNA
aggtaaacaagtttcaagac

SEQ ID NO: 16 - DNA, 20 bp, synthetic construct
  source 1..20: mol_type = other DNA
taaggtaaacaagtttcaag

SEQ ID NO: 17 - DNA, 20 bp, synthetic construct
  source 1..20: mol_type = other DNA
taattttaaggtaaacaagt

SEQ ID NO: 18 - DNA, 20 bp, synthetic construct
  source 1..20: mol_type = other DNA
aacactgagattctgataat

SEQ ID NO: 19 - DNA, 20 bp, synthetic construct
  source 1..20: mol_type = other DNA
agtactttcaaacactgaga

SEQ ID NO: 20 - DNA, 20 bp, synthetic construct
  source 1..20: mol_type = other DNA
atatgtttgtgcttcagtac

SEQ ID NO: 21 - DNA, 20 bp, synthetic construct
  source 1..20: mol_type = other DNA
gatatatgtttgtgcttcag

SEQ ID NO: 22 - DNA, 20 bp, synthetic construct
  source 1..20: mol_type = other DNA
gtacagagatgatatatgtt

SEQ ID NO: 23 - DNA, 18 bp, synthetic construct
  source 1..18: mol_type = other DNA
gggtagggactggaatga

SEQ ID NO: 24 - DNA, 20 bp, synthetic construct
  source 1..20: mol_type = other DNA
gtagggactggaatgaagat

SEQ ID NO: 25 - DNA, 20 bp, synthetic construct
  source 1..20: mol_type = other DNA
gggtagggactggaatgaag

SEQ ID NO: 26 - RNA, 16 nt, synthetic construct
  source 1..16: mol_type = other RNA
ccgcgctagggaccga

SEQ ID NO: 27 - RNA, 19 nt, synthetic construct
  source 1..19: mol_type = other RNA
ccgcagccgcgctagggac

SEQ ID NO: 28 - RNA, 20 nt, synthetic construct
  source 1..20: mol_type = other RNA
cgcagctctccgccccgcag

SEQ ID NO: 29 - RNA, 19 nt, synthetic construct
  source 1..19: mol_type = other RNA
gtgggcgcgctgggccagc

SEQ ID NO: 30 - RNA, 18 nt, synthetic construct
  source 1..18: mol_type = other RNA
ctcctcaggtgggcgcgc

SEQ ID NO: 31 - RNA, 19 nt, synthetic construct
  source 1..19: mol_type = other RNA
ccgcctcctcaggtgggcg

SEQ ID NO: 32 - RNA, 18 nt, synthetic construct
  source 1..18: mol_type = other RNA
tgcggaccccgccgcctc

SEQ ID NO: 33 - RNA, 18 nt, synthetic construct
  source 1..18: mol_type = other RNA
gacgcctgcggaccccgc

SEQ ID NO: 34 - RNA, 17 nt, synthetic construct
  source 1..17: mol_type = other RNA
cgacgcctgcggacccc

SEQ ID NO: 35 - RNA, 17 nt, synthetic construct
  source 1..17: mol_type = other RNA
cgcgacgcctgcggacc

SEQ ID NO: 36 - RNA, 20 nt, synthetic construct
  source 1..20: mol_type = other RNA
gtcccgcgacgcctgcggac

SEQ ID NO: 37 - RNA, 18 nt, synthetic construct
  source 1..18: mol_type = other RNA
tcccgcgacgcctgcgga

SEQ ID NO: 38 - RNA, 19 nt, synthetic construct
  source 1..19: mol_type = other RNA
gtcccgcgacgcctgcgga

SEQ ID NO: 39 - RNA, 20 nt, synthetic construct
  source 1..20: mol_type = other RNA
cgtcccgcgacgcctgcgga

SEQ ID NO: 40 - RNA, 17 nt, synthetic construct
  source 1..17: mol_type = other RNA
tcccgcgacgcctgcgg

SEQ ID NO: 41 - RNA, 19 nt, synthetic construct
  source 1..19: mol_type = other RNA
cgtcccgcgacgcctgcgg

SEQ ID NO: 42 - RNA, 20 nt, synthetic construct
  source 1..20: mol_type = other RNA
tcgtcccgcgacgcctgcgg

SEQ ID NO: 43 - RNA, 18 nt, synthetic construct
  source 1..18: mol_type = other RNA
cgtcccgcgacgcctgcg

SEQ ID NO: 44 - RNA, 17 nt, synthetic construct
  source 1..17: mol_type = other RNA
tcgtcccgcgacgcctg

SEQ ID NO: 45 - RNA, 17 nt, synthetic construct
  source 1..17: mol_type = other RNA
ccgatctcctcgtcccg

SEQ ID NO: 46 - RNA, 17 nt, synthetic construct
  source 1..17: mol_type = other RNA
gtctcccggctccgatc

SEQ ID NO: 47 - RNA, 18 nt, synthetic construct
  source 1..18: mol_type = other RNA
gagtctcccggctccgat

SEQ ID NO: 48 - RNA, 18 nt, synthetic construct
  source 1..18: mol_type = other RNA
cgctgcgcgagtctcccg

SEQ ID NO: 49 - RNA, 18 nt, synthetic construct
  source 1..18: mol_type = other RNA
ggcgctgcgcgagtctcc

SEQ ID NO: 50 - RNA, 16 nt, synthetic construct
  source 1..16: mol_type = other RNA
ccgcgctagggaccga

SEQ ID NO: 51 - RNA, 19 nt, synthetic construct
  source 1..19: mol_type = other RNA
ccgcagccgcgctagggac

SEQ ID NO: 52 - RNA, 20 nt, synthetic construct
  source 1..20: mol_type = other RNA
cgcagctctccgccccgcag

SEQ ID NO: 53 - RNA, 19 nt, synthetic construct
  source 1..19: mol_type = other RNA
gtgggcgcgctgggccagc

SEQ ID NO: 54 - RNA, 18 nt, synthetic construct
  source 1..18: mol_type = other RNA
ctcctcaggtgggcgcgc

SEQ ID NO: 55 - RNA, 19 nt, synthetic construct
  source 1..19: mol_type = other RNA
ccgcctcctcaggtgggcg

SEQ ID NO: 56 - RNA, 18 nt, synthetic construct
  source 1..18: mol_type = other RNA
tgcggaccccgccgcctc

SEQ ID NO: 57 - RNA, 18 nt, synthetic construct
  source 1..18: mol_type = other RNA
gacgcctgcggaccccgc

SEQ ID NO: 58 - RNA, 17 nt, synthetic construct
  source 1..17: mol_type = other RNA
cgacgcctgcggacccc

SEQ ID NO: 59 - RNA, 17 nt, synthetic construct
  source 1..17: mol_type = other RNA
cgcgacgcctgcggacc

SEQ ID NO: 60 - RNA, 20 nt, synthetic construct
  source 1..20: mol_type = other RNA
gtcccgcgacgcctgcggac

SEQ ID NO: 61 - RNA, 18 nt, synthetic construct
  source 1..18: mol_type = other RNA
tcccgcgacgcctgcgga

SEQ ID NO: 62 - RNA, 19 nt, synthetic construct
  source 1..19: mol_type = other RNA
gtcccgcgacgcctgcgga

SEQ ID NO: 63 - RNA, 20 nt, synthetic construct
  source 1..20: mol_type = other RNA
cgtcccgcgacgcctgcgga

SEQ ID NO: 64 - RNA, 17 nt, synthetic construct
  source 1..17: mol_type = other RNA
tcccgcgacgcctgcgg

SEQ ID NO: 65 - RNA, 19 nt, synthetic construct
  source 1..19: mol_type = other RNA
cgtcccgcgacgcctgcgg

SEQ ID NO: 66 - RNA, 20 nt, synthetic construct
  source 1..20: mol_type = other RNA
tcgtcccgcgacgcctgcgg

SEQ ID NO: 67 - RNA, 18 nt, synthetic construct
  source 1..18: mol_type = other RNA
cgtcccgcgacgcctgcg

SEQ ID NO: 68 - RNA, 17 nt, synthetic construct
  source 1..17: mol_type = other RNA
tcgtcccgcgacgcctg

SEQ ID NO: 69 - RNA, 17 nt, synthetic construct
  source 1..17: mol_type = other RNA
ccgatctcctcgtcccg

SEQ ID NO: 70 - RNA, 17 nt, synthetic construct
  source 1..17: mol_type = other RNA
gtctcccggctccgatc

SEQ ID NO: 71 - RNA, 18 nt, synthetic construct
  source 1..18: mol_type = other RNA
gagtctcccggctccgat

SEQ ID NO: 72 - RNA, 18 nt, synthetic construct
  source 1..18: mol_type = other RNA
cgctgcgcgagtctcccg

SEQ ID NO: 73 - RNA, 18 nt, synthetic construct
  source 1..18: mol_type = other RNA
ggcgctgcgcgagtctcc

SEQ ID NO: 74 - DNA, 19 bp, synthetic construct
  source 1..19: mol_type = other DNA
tatttggatgagagcaggc

SEQ ID NO: 75 - DNA, 20 bp, synthetic construct
  source 1..20: mol_type = other DNA
ttatttggatgagagcaggc

SEQ ID NO: 76 - DNA, 20 bp, synthetic construct
  source 1..20: mol_type = other DNA
gttatttggatgagagcagg

SEQ ID NO: 77 - DNA, 20 bp, synthetic construct
  source 1..20: mol_type = other DNA
ggttatttggatgagagcag

SEQ ID NO: 78 - DNA, 21 bp, synthetic construct
  source 1..21: mol_type = other DNA
ggtagggactggaatgaagat

SEQ ID NO: 79 - DNA, 21 bp, synthetic construct
  source 1..21: mol_type = other DNA
gggtagggactggaatgaaga

SEQ ID NO: 80 - DNA, 21 bp, synthetic construct
  source 1..21: mol_type = other DNA
ggttatttggatgagagcagg

SEQ ID NO: 81 - DNA, 22 bp, synthetic construct
  source 1..22: mol_type = other DNA
gggtagggactggaatgaagat

SEQ ID NO: 82 - DNA, 22 bp, synthetic construct
  source 1..22: mol_type = other DNA
aggttatttggatgagagcagg

SEQ ID NO: 83 - DNA, 22 bp, synthetic construct
  source 1..22: mol_type = other DNA
caggttatttggatgagagcag

SEQ ID NO: 84 - DNA, 24 bp, synthetic construct
  source 1..24: mol_type = other DNA
ttatttggatgagagcaggcactg

SEQ ID NO: 85 - DNA, 20 bp, synthetic construct
  source 1..20: mol_type = other DNA
ccattcatcctaacagtcca

SEQ ID NO: 86 - DNA, 20 bp, synthetic construct
  source 1..20: mol_type = other DNA
tccatacccaagcaatgtgc

SEQ ID NO: 87 - DNA, 20 bp, synthetic construct
  source 1..20: mol_type = other DNA
tccaagaacacagacatctc

SEQ ID NO: 88 - DNA, 20 bp, synthetic construct
  source 1..20: mol_type = other DNA
ggcactgtgacttagactgg

SEQ ID NO: 89 - DNA, 20 bp, synthetic construct
  source 1..20: mol_type = other DNA
cctccagaacccatctgttc

SEQ ID NO: 90 - DNA, 20 bp, synthetic construct
  source 1..20: mol_type = other DNA
gactaatctcagtgcaaggg

SEQ ID NO: 91 - DNA, 20 bp, synthetic construct
  source 1..20: mol_type = other DNA
ggtcctgaagcatgagcact

SEQ ID NO: 92 - DNA, 20 bp, synthetic construct
  source 1..20: mol_type = other DNA
gaactgaggcgggcggtggt

SEQ ID NO: 93 - DNA, 20 bp, synthetic construct
  source 1..20: mol_type = other DNA
gagggcatcactgaacacga

SEQ ID NO: 94 - DNA, 20 bp, synthetic construct
  source 1..20: mol_type = other DNA
ggcgtccattcatcctaaca

SEQ ID NO: 95 - DNA, 20 bp, synthetic construct
  source 1..20: mol_type = other DNA
aggcgtccattcatcctaac

SEQ ID NO: 96 - DNA, 20 bp, synthetic construct
  source 1..20: mol_type = other DNA
gggttatgtacaaggtcaca

SEQ ID NO: 97 - DNA, 20 bp, synthetic construct
  source 1..20: mol_type = other DNA
agggttatgtacaaggtcac

SEQ ID NO: 98 - DNA, 20 bp, synthetic construct
  source 1..20: mol_type = other DNA
ctaccgttccatacccaagc

SEQ ID NO: 99 - DNA, 20 bp, synthetic construct
  source 1..20: mol_type = other DNA
ggacaggaacaaccccaaac

SEQ ID NO: 100 - DNA, 20 bp, synthetic construct
  source 1..20: mol_type = other DNA
cctcagtgtgaaatactcca

SEQ ID NO: 101 - DNA, 20 bp, synthetic construct
  source 1..20: mol_type = other DNA
cgtgcagacaccccagcctc

SEQ ID NO: 102 - DNA, 20 bp, synthetic construct
  source 1..20: mol_type = other DNA
cttgaggatggcgagacagc

SEQ ID NO: 103 - DNA, 20 bp, synthetic construct
  source 1..20: mol_type = other DNA
gaggtcaagggcggcgaggg

SEQ ID NO: 104 - DNA, 20 bp, synthetic construct
  source 1..20: mol_type = other DNA
ggaggtcaagggcggcgagg

SEQ ID NO: 105 - DNA, 20 bp, synthetic construct
  source 1..20: mol_type = other DNA
aacaccacaatgcagcgagc

SEQ ID NO: 106 - DNA, 20 bp, synthetic construct
  source 1..20: mol_type = other DNA
atcaggtcgtataagttggg

SEQ ID NO: 107 - DNA, 20 bp, synthetic construct
  source 1..20: mol_type = other DNA
gggacagaacaagcagcggg

(19) United States
(12) Patent Application Publication
(10) Pub. No.: US 2023/0212567 A1
(43) Pub. Date: Jul. 6, 2023

(54) THERAPEUTICS FOR HAPLOINSUFFICIENCY CONDITIONS

(71) Applicant: Q-STATE BIOSCIENCES, INC., Cambridge, MA (US)

(72) Inventors: Graham T. Dempsey, Sudbury, MA (US); Caitlin Lewarch, Cambridge, MA (US); Matt Mccabe, Cambridge, MA (US); James Fink, Cambridge, MA (US); Luis Williams, Cambridge, MA (US); David Gerber, Cambridge, MA (US); Sudhir Agrawal, Cambridge, MA (US)

(21) Appl. No.: 17/934,428

(22) Filed: Sep. 22, 2022

Related U.S. Application Data
(60) Provisional application No. 63/247,783, filed on Sep. 23, 2021.

(51) Int. Cl. C12N 15/113

(52) U.S. Cl. CPC C12N 15/113; C12N 2310/11; C12N 2310/321; C12N 2310/315

(57) ABSTRACT

The invention relates to therapeutic compositions for disorders associated with haploinsufficiency. The invention provides antisense oligonucleotides useful for treating early-onset epileptic encephalopathy by promoting expression of Syntaxin-binding protein 1 (STXBP1). The invention provides compositions that include synthetic antisense oligonucleotides (ASOs) that prevent certain miRNAs from interfering with production of the STXBP1 protein or bind to the 5′-UTR of the STXBP1 transcript and augment translation of the STXBP1 protein. When the composition is delivered to a patient with STXBP1 haploinsufficiency, the ASOs prevent miRNA from downregulating synthesis of STXBP1 protein.

### TECHNICAL FIELD

The invention relates to therapeutic compositions for disorders associated with haploinsufficiency.

### SEQUENCE LISTING

This application includes and incorporates by reference the electronic sequence listing in ST.26 format, filed herewith. The sequence listing, created on Sep. 22, 2022 is entitled “QSTA-044-01US-Sequece-Listing.xml”, and is 98 kilobytes in size.

### BACKGROUND

Early onset epileptic encephalopathies are devastating, and often fatal, conditions characterized by intellectual disability and cerebral dysfunction associated with severe epileptic activity and eventual cognitive, sensory, and motor function deterioration. Not only do such conditions vary in onset, outcome, and severity, those epilepsies commonly present as refractory epilepsy, meaning that they do not respond to antiepileptic drugs. Infants born with refractory early-onset epilepsy thus have very poor prognosis.

Symptoms of refractory early-onset epilepsy include severe language impairment, difficulties managing social interactions, fine motor difficulties, hyperactivity, ataxia and tremor, and autistic features. There are limited options for epilepsy patients who do not respond to antiepileptics. Some success has been reported with modified diets or electrical neuromodulation. However, not all cases are treatable, and some people are faced with the challenge of surviving with the condition.

### SUMMARY

The invention provides antisense oligonucleotides useful for treating early-onset epileptic encephalopathy by promoting expression of Syntaxin-binding protein 1 (STXBP1). Some individuals are born with a heterozygous loss-of-function mutation on the STXBP1 gene that encodes STXBP1, a condition known as haploinsufficiency of STXBP1. In haploinsufficiency of STXBP1, there is only one fully functional copy of the STXBP1 gene. During neurodevelopment, all STXBP1 protein must be produced by transcribing that one copy of the gene to pre-mRNA, splicing the pre-mRNA to mRNA, and translating the mRNA to protein. The invention makes use of one insight that micro-RNAs (miRNAs) may play a role in gene regulation and interfere with the production of STXBP1 protein. Specifically, certain miRNAs may bind to STXBP1 RNA and prevent protein production. Embodiments of the invention provide compositions that include synthetic antisense oligonucleotides (ASOs) that prevent certain miRNAs from interfering with production of the STXBP1 protein. The invention also makes use of an insight that features of the STXBP1 mRNA may impede translational efficiency. Specifically, the 5′ untranslated region (5′UTR) of the STXBP1 mRNA may form stable hairpins that inhibit recruitment of the translational machinery. Additionally, the STXBP1 mRNA may include upstream open reading frames (uORFs) where translation may initiate, inhibiting initiation at the downstream primary open reading frame. Some embodiments of the invention provide compositions that include ASOs that destabilize hairpins and/or mask uORFs to thereby improve translation of the STXBP1 protein. When delivered to a patient with STXBP1 haploinsufficiency, ASOs of the invention prevent miRNA from downregulating synthesis of STXBP1 protein and/or prevent hairpins or uORFs from impeding translational efficiency.

Where otherwise untreated STXBP1 haploinsufficiency may lead to a deficit of the expressed protein during neurodevelopment, treatment with a composition of the invention increases production of functional STXBP1 protein from the non-mutant allele. The increase of STXBP1 protein results in a healthy phenotype despite the haploinsufficient genotype. Thus, compositions of the invention are useful to treat or prevent the development of early-onset epilepsy or its symptoms and related conditions. Treatment may be delivered upon detection of any symptoms or on detection, e.g., by genetic screening, of the haploinsufficiency.

Without being bound by any mechanism of action, it may be that miRNAs bind to sequences within a pre-mRNA or mRNA, such as a 3′ UTR of an mRNA or a 3′ regulatory region. This sequence-specific binding may induce translational repression, RNA cleavage, mRNA deadenylation, or mRNA decapping. In compositions of the invention, ASOs include a nucleotide sequence that may either bind the ASO to the STXBP1 mRNA or to the miRNA and prevent or inhibit miRNA-mediated translational repression. Preferably the ASOs bind to the STXBP1 mRNA and may sterically block the miRNA from binding to its normal target site. In one aspect, the invention comprises blocking the miRNA derived from the 491, 424, 423, 338, 219, 218, 154, 143, 141, 30, and 1 pre-miRNAs. Certain ASOs of the invention are designed to specifically block the interaction of STXBP1 mRNA with miRNA such as, for example, any of miR-491-5p, miR-424-5p, miR-423-5p, miR-423-3p, miR-338-3p, miR-30b-5p, miR-219a-5p, miR-218-5p, miR-154-5p, miR-143-3p, miR-141-3p, and miR-1-3p. An insight of the invention is that the ASO does not need to have perfect identity with the sequence of, or the reverse complement of the sequence of, either of the miRNA or the binding location of the miRNA on the STXBP1 pre-RNA or mRNA.

In one aspect, the effect of an miRNA is manifest when a seed region of 7 to 8 nucleotides (or possibly as short as 5 or 6 bases) in the miRNA matches a cognate sequence in the target. In one aspect, the miRNA and mRNA target matching is not one-to-one, but one-to-many and/or many-to-one, meaning that one miRNA may regulate multiple mRNAs and one mRNA may be regulated by multiple miRNAs. Targets of the invention may be those miRNAs for which blocking binding of the miRNA provides for the up-regulation of STXBP1 protein and effective treatment of STXBP1 haploinsufficiency. Because it may be that only a short seed region is necessary for miRNA effect and that selecting miRNAs to target is a challenge, some ASOs with only limited sequence identity to the right miRNA or its reverse complement are useful in treating STXBP1 haploinsufficiency. Thus, the disclosure provides methods and compositions invented and discovered to be useful in treating conditions such as early-onset epilepsy or its symptoms and in which the new usefulness may lie at least in-part in the specified miRNA targets disclosed and addressed by compositions of the invention.

In other aspects, methods and compositions of the disclosure operate by providing ASOs that destabilize 5′UTR hairpins and/or masking cryptic ORFs such as an uORF. Compositions of the invention may include at least one nucleic acid that promotes expression of Syntaxin binding protein 1 (STXBP1) and has at least 50% sequence similarity to one of SEQ ID Nos: 26-73. Preferably, the nucleic acid has a sequence substantially identical to one of SEQ ID NO: 26 through SEQ ID NO: 73 such as at least 90% identical or greater, albeit with 2′-O-Methyl on the ribose sugars. While phosphorothioate linkages may be included, ASOs of these aspects may preferably have mostly or entirely phosphodiester (PO) linkages. ASOs of these aspects may operate by destabilizing hairpins in the 5′UTR of the STXBP1 mRNA, by masking an uORF, or both. Specifically, ASOs based on SEQ ID Nos: 26-73 may function by destabilizing hairpins in the 5′UTR of the STXBP1 mRNA. ASOs based on SEQ ID Nos: 27-31, 45-47, 51-55, and 69-71 may operate by destabilizing hairpins in the 5′UTR of the STXBP1 mRNA and by masking an uORF.

Compositions of the invention may include at least one nucleic acid that promotes expression of Syntaxin binding protein 1 (STXBP1) and has at least 50% sequence similarity to one of SEQ ID Nos: 74-107. Preferably, the nucleic acid has a sequence substantially identical to one of SEQ ID NO: 74 through SEQ ID NO: 107 such as at least 90% identical or greater, albeit with a majority of the bases in the nucleic acid having 2′-O-methoxyethyl (2′-MOE) ribose sugars and/or a majority inter-base linkages in the nucleic acid having phosphorothioate bonds, though, ASOs of these aspects may have phosphodiester (PO) linkages. ASOs of these aspects may operate by destabilizing and/or masking a 3′ regulatory region of the STXBP1 mRNA, and/or by masking an uORF. Specifically, ASOs based on SEQ ID Nos: 85-107 may function by via interaction with a 3′ regulatory region of the STXBP1 mRNA.

In certain aspects, the invention provides a composition that includes at least one nucleic acid that promotes expression of Syntaxin binding protein 1 (STXBP1) and has at least 25% sequence similarity to one of SEQ ID Nos: 1-3, 7-13, 15-18, and 23-107, at least 60% sequence similarity to one of SEQ ID NOS: 1-3, 7-13, 15-19, and 23-107, at least 70% sequence similarity to one of SEQ ID NOS: 1-3, 7-13, 15-19, and 22-107, at least 75% sequence similarity to one of SEQ ID NOS: 1-3, 6-13, 15-19, and 22-107, at least 80% sequence similarity to one of SEQ ID NOS: 1-3, 6-13, 15-19, and 21-107, at least 85% sequence similarity to one of SEQ ID NOS: 1-3, and 6-107, and/or at least 90% sequence similarity to one of SEQ ID NOS: 1-107. Preferably, the nucleic acid includes a contiguous stretch of at least about 4 to 6 bases with at least 80% sequence similarity (in the contiguous stretch) to a corresponding contiguous region in the one of SEQ ID Nos: 1-107. Preferably, the contiguous stretch matches the corresponding stretch in the given sequence 100% and more preferably the contiguous matching stretch is about 4 or 5 or 6 or 7 or 8 or 9 bases long. The contiguous stretch preferably corresponds to a seed region by which the associated miRNA would initially bind to a STXBP1 RNA (such as pre-mRNA or mRNA). In embodiments, the nucleic acid has a length between about 5 and 50 bases and the nucleic acid has a region of at least 5 contiguous bases with a 100% match to a segment within one of SEQ ID Nos: 1-25. The nucleic acid may include at least about 50% RNA bases with a 2′ modification on a ribose sugar. At least about 50% of the inter-base linkages in the nucleic acid may not be phosphodiester (PO) bonds. Any or all of the linkages may be PO or phosphorothioate (PS). In some embodiments at least about 12 contiguous bases in the nucleic acid have at least 90% sequence identity to a corresponding 12 contiguous bases in one of SEQ ID Nos: 1-25. Preferably a majority of the bases of the nucleic acid have a 2′-O-methoxy-ethyl-modified ribose, 2′-OMe, or a combination thereof. A majority of inter-base linkages in the nucleic acid may be phosphorothioate (PS) bonds. In certain embodiments, all of the bases in the nucleic acid comprise 2′-O-methoxyethyl ribose sugars and/or all bonds are PS.

In some embodiments, the nucleic acid has at least 90% sequence similarity to one of SEQ ID Nos: 1-25 and all of the bases in the nucleic acid comprise 2′-O-methoxy-ethyl ribose sugars. The nucleic acid may have at least 94% sequence similarity to one of SEQ ID Nos: 1-25 with all of the bases in the nucleic acid being 2′-O-methoxyethyl ribose sugars. In certain embodiments at least about 90% of inter-base linkages in the nucleic acid are phosphorothioate bonds. The nucleic acid may have 100% sequence similarity to one of SEQ ID Nos: 1-25; all of the bases in the nucleic acid may be 2′-O-methoxyethyl ribose sugars; and all inter-base linkages in the nucleic acid may be phosphorothioate bonds.

Compositions of the invention may include one or any combination of nucleic acids with the following features (a) through (l): (a) the nucleic acid hybridizes to a binding site of, and blocks binding of an miRNA (such as miR-423-3p) and the nucleic acid has at least 75% sequence similarity to one selecting from the group consisting of SEQ ID Nos: 1, 2, and 3; (b) the nucleic acid hybridizes to a binding site of, and blocks binding of an miRNA (such as mirR-491-5p) and the nucleic acid has at least 90% sequence similarity to one selecting from the group consisting of SEQ ID Nos: 4 and 5; (c) the nucleic acid hybridizes to a binding site of, and blocks binding of an miRNA such as miR-338-3p and the nucleic acid has at least 75% sequence similarity to SEQ ID NO: 6; (d) the nucleic acid hybridizes to a binding site of, and blocks binding of an miRNA (such as miR-1-3p) and the nucleic acid has at least 75% sequence similarity to one selecting from the group consisting of SEQ ID Nos: 7, 8, 9, 23, 24, and 25; (e) the nucleic acid hybridizes to a binding site of, and blocks binding of an miRNA (such as miR-423-5p) and the nucleic acid has at least 75% sequence similarity to SEQ ID NO: 10; (f) the nucleic acid hybridizes to a binding site of, and blocks binding of an miRNA (such as miR 5p) and the nucleic acid has at least 75% sequence similarity to one selecting from the group consisting of SEQ ID Nos: 11 and 12; (g) the nucleic acid hybridizes to a binding site of, and blocks binding of an miRNA (such as miR-219a-5p) and the nucleic acid has at least 75% sequence similarity to SEQ ID NO: 13; (h) the nucleic acid hybridizes to a binding site of, and blocks binding of an miRNA (such as mirR-424-5p) and the nucleic acid has at least 85% sequence similarity to SEQ ID NO: 14; (i) the nucleic acid hybridizes to a binding site of, and blocks binding of an miRNA (such as miR-30b-5p) and the nucleic acid has at least 75% sequence similarity to one selecting from the group consisting of SEQ ID Nos: 15, 16, and 17; (j) the nucleic acid hybridizes to a binding site of, and blocks binding of an miRNA (such as miR-141-3p) and the nucleic acid has at least 75% sequence similarity to one selecting from the group consisting of SEQ ID No: 18 and 19; (k) the nucleic acid hybridizes to a binding site of, and blocks binding of an miRNA (such as miR-218-5p) and the nucleic acid has at least 85% sequence similarity to SEQ ID No: 20 or the nucleic acid has at least 80% sequence similarity to SEQ ID NO: 21; and (1) the nucleic acid hybridizes to a binding site of, and blocks binding of an miRNA (such as miR-143-3p) and the nucleic acid has at least 75% sequence similarity to SEQ ID NO: 22. In this paragraph, any appearance of “75%”, “80%”, “85%”, and “90%” may be replaced with a higher value “85%”, 95%, or even “99%”, depending, to state successively more preferred embodiments.

In certain steric blocking oligonucleotide (SBO) embodiments, the nucleic acid has 100% sequence similarity to the one of SEQ ID Nos: 1-25 and 74-84 with, e.g., a majority of the bases in the nucleic acid having 2′-O-methoxyethyl (2′-MOE) ribose sugars and/or a majority inter-base linkages in the nucleic acid having phosphorothioate bonds. The nucleic acid may have any combination of modified sugars, e.g., 2′-MOE and/or 2′-O-Methyl and/or any combination of inter-base linkages, e.g., PS/PO in any combination in the backbone. Optionally all of the bases in the nucleic acid comprise 2′-O-methoxyethyl ribose sugars and all inter-base linkages in the nucleic acid are phosphorothioate bonds. Any of the nitrogenous bases may be methylated, i.e., 5-methylcytosine (mC) and/or 5-methyluridine (mU) (which is T).

In other UTR/ORF embodiments, the nucleic acid has 100% sequence similarity to one of SEQ ID Nos: 26-73 with, e.g., a majority of the bases in the nucleic acid having 2′-O-methyl ribose sugars and/or a majority inter-base linkages in the nucleic acid having phosphodiester bonds. The nucleic acid may have any combination of modified sugars, e.g., 2′-MOE and/or 2′-OMe and/or any combination of inter-base linkages, e.g., PS/PO in any combination in the backbone. Optionally all of the bases in the nucleic acid comprise 2′-OMe ribose sugars. Nitrogenous bases may be methylated, i.e., mC, mU (which is T). For SEQ ID Nos: 26-49, all inter-base linkages in the nucleic acid are phosphodiester bonds. For SEQ ID Nos: 50-73, all inter-base linkages in the nucleic acid are phosphorothioate bonds.

In other embodiments targeting 3′ regulatory regions, the nucleic acid has 100% sequence similarity to one of SEQ ID Nos: 85-107, e.g., a majority of the bases in the nucleic acid having 2′-O-methoxy-ethyl (2′-MOE) ribose sugars and/or a majority inter-base linkages in the nucleic acid having phosphorothioate bonds. The nucleic acid may have any combination of modified sugars, e.g., 2′-MOE and/or 2′-O-Methyl and/or any combination of inter-base linkages, e.g., PS/PO in any combination in the backbone. Optionally all of the bases in the nucleic acid comprise 2′-O-methoxyethyl ribose sugars and all inter-base linkages in the nucleic acid are phosphorothioate bonds. Any of the nitrogenous bases may be methylated, i.e., 5-methylcytosine (mC) and/or 5-methyluridine (mU) (which is T).

Related aspects provide methods of treating early onset epileptic encephalopathy. Methods include delivering to a patient in need thereof one of the compositions described above. Preferably the composition is delivered across the blood-brain barrier. The composition may be delivered by intrathecal injection. The delivering step leads to increased expression of Syntaxin binding protein 1 (STXBP1) in the patient. Methods may include selecting the patient by identifying a heterozygous loss-of-function mutation in a STXBP1 gene.

### BRIEF DESCRIPTION OF THE DRAWINGS

FIG. 1 shows results from screening of 21 ASOs.

FIG. 2 shows dose response of certain ASOs in fibroblasts.

FIG. 3 shows dose response of certain ASOs.

FIG. 4 shows results from screening of 21 STXBP1 miRNA-blocking ASOs in iPSC derived NGN2 neurons, 7-days post-treatment.

FIG. 5 shows results from screening certain ASOs.

FIG. 6 shows results from screening certain ASOs.

FIG. 7 shows results from screening.

FIG. 8 shows results from screening an ASO.

FIG. 9 shows results from Screening of 8 STXBP1 miRNA-blocking ASOs: SH-SY5Y Neuroblastoma Cells (48 hours post-treatment).

FIGS. 10-11 show images of Western blot gels that provide the results of an example screening of STXBP1 protein boosting with miRNA-blocking ASOs in human iPSC-derived NGN2 neurons.

FIGS. 12-13 provide a summary of screening for boosting of STXBP1 protein across several 3′ miRNA-targeting ASOs in human iPSC-derived NGN2 neurons at 2 timepoints in culture.

FIG. 14 provides results showing that STXBP1 ASO hits modulate STXBP1 protein in dose-response in human iPSC-derived NGN2 neurons.

FIG. 15 shows quantification of 5′ STXBP1 ASOs screened in human iPSC-derived neurons with Western Blotting revealing STXBP1 protein boosting for several ASOs of the invention.

FIGS. 16-17 provide results showing identification of an all-optical electrophysiological synaptic cellular phenotype using the BRITE™ System by Q-State Biosciences, Inc.

FIG. 18 shows synaptic phenotype rescue by re-introduction of STXBP1 gene via lentiviral delivery.

### DETAILED DESCRIPTION

The invention provides antisense oligonucleotides useful for treating early-onset epileptic encephalopathy by promoting expression of Syntaxin-binding protein 1 (STXBP1). Some individuals are born with a heterozygous loss-of-function mutation on the STXBP1 gene that encodes STXBP1, a condition known as haploinsufficiency of STXBP1. The invention makes use of the insight that micro-RNAs (miRNAs) may play a role in gene regulation that interferes with the production of STXBP1 protein. Specifically, it is thought that certain miRNAs may bind to STXBP1 RNA and prevent protein production. The invention provides compositions that include synthetic antisense oligonucleotides (ASOs) that prevent certain miRNAs from interfering with production of the STXBP1 protein. When the composition is delivered to a patient with STXBP1 haploinsufficiency, the ASOs prevent miRNA from downregulating synthesis of STXBP1 protein.

Compositions and methods of the invention preferably use at least one nucleic acid that promotes expression of Syntaxin Binding Protein 1 and has at least 25% sequence identity or similarity to one of SEQ ID Nos: 1-3, 7-13, 15-18, and 23-107, at least 60% sequence identity or similarity to one of SEQ ID NOS: 1-3, 7-13, 15-19, and 23-107, at least 70% sequence identity or similarity to one of SEQ ID NOS: 1-3, 7-13, 15-19, and 22-107, at least 75% sequence identity or similarity to one of SEQ ID NOS: 1-3, 6-13, 15-19, and 22-107, at least 80% sequence identity or similarity to one of SEQ ID NOS: 1-3, 6-13, 15-19, and 21-107, at least 85% sequence identity or similarity to one of SEQ ID NOS: 1-3, and 6-107, and/or at least 90% sequence identity or similarity to one of SEQ ID NOS: 1-107. In preferred aspects, the nucleic acid has at least 25% sequence identity or similarity to one of SEQ ID NOS: 1-3, 7-13, 15-19, and 23-25, at least 60% sequence identity or similarity to SEQ ID NO: 19, at least 70% sequence identity or similarity to SEQ ID NO: 22, at least 75% sequence identity or similarity to SEQ ID NO: 6, at least 80% sequence identity or similarity to SEQ ID NO: 21, at least 85% sequence identity or similarity to one of SEQ ID NOS: 14 and 21, at least 90% sequence identity or similarity to one of SEQ ID NOS: 4 and 5. Preferably, the nucleic acid includes a contiguous stretch of at least about 6 bases with at least 80% sequence similarity to a corresponding contiguous region in the one of SEQ ID Nos: 1-25. Preferably, the contiguous stretch matches the corresponding stretch in the given sequence 100% and more preferably the contiguous matching stretch is about 4 or 5 or 6 or 7 or 8 or 9 bases long.

The STXBP1 gene is located on human chromosome 9 and encodes a protein essential for presynaptic neurotransmitter release, Syntaxin Binding Protein 1 (STXBP1). Heterozygous loss-of-function mutations in the STXBP1 gene result in Early-onset Infantile Epileptic Encephalopathy Type IV (EIEE4), also known as STXBP1-encephalopathy, a disorder characterized by severe seizures and intellectual disability. STXBP1-encephalopathy is the third most common genetic epilepsy (˜2000 known patients in the US) with an estimated incidence of 1:30,000. The invention provides for ASO-mediated boosting of expression from the unaffected allele as an approach for correcting haploinsufficiency disorders such as STXBP1-encephalopathy. STXBP1-encephalopathy is generally caused by heterozygous loss-of-function mutations in the STXBP1 gene, comprising a known disease mechanism. STXBP1-encephalopathy patients typically experience poor outcomes with typical anti-epileptic regimens (commonly used anti-epileptic drugs are phenobarbital, valproic acid, and vigabatrin). The invention provides a new, different approach with compositions and methods that may be used in treatments that address the root cause of the disease and have potential for disease modification. ASO-mediated boosting of STXBP1 expression from the unaffected allele directly addresses the genetic mechanism of the disease.

Compositions of the invention and their effects may be assessed with an Optopatch assay. Generally, Optopatch includes the use of in vitro neurons that include optogenetic constructs that provide neural activation under optical stimulus (e.g., a modified algal channelrhodopsin that causes the neuron to fire in response to light) and optical reporters of neural activity (modified archaerhodopsins that emit light in proportion to neuronal membrane voltage and yield signals of neuronal activity). The in vitro neurons may be assayed in a fluorescence microscopy instrument, and may also be (e.g., subsequently) evaluated by e.g., staining (e.g., immunocytochemistry), RNA-Seq, or other such assay. Any suitable optogenetic constructs, optogenetic microscope, or other assays may be used. For example, suitable optogenetic constructs include those described in U.S. Pat. No. 9,594,075, incorporated by reference. Suitable optogenetic microscopes include those described in U.S. Pat. No. 10,288,863, incorporated by reference. Compositions of the invention and their effects may be assessed using iPSC-derived neuronal cell lines with mutations in the STXBP1 gene (both heterozygous and homozygous loss-of-function mutations). Optopatch phenotyping is being performed on such cell lines.

To provide composition and methods of the invention, sequences for ASOs may be selected by a process that includes balancing various factors such as: 1. determine target transcript regions and generate sequences (all possible N-mers); 2. exclusion based on experimental constraints (isoforms, homology); 3. exclusion based on predicted off-target mRNA, pre-mRNA, miRNA, and lncRNA hits in humans and non-human primates (rhesus and cynomolgus macaque); 4. exclusion/filtering based on sequence characteristics (tetra-G, tetra-C, CpG, palindromes, GC content, and poly-X stretches); 5. filter based on thermodynamic parameters (Tm, hairpin ΔG, ASO duplex ΔG, ASO:target ΔG); and 7. choose ASOs within regions based on thermodynamic parameters, spacing, and experimental goals (Overall ΔG). To determine target transcript regions, criteria may be applied from literature, sequencing-based assays, and models to identify miRNA binding sites to block. For targets passing the criteria, 7-8 nt predicted miRNA binding sites are identified based on seed sequence and a model that includes a variety of contextual information. Conserved sites are more likely to be functional/relevant.

Identifying targets to address with compositions of the invention may be performed using any suitable technique or combination of techniques. Preferred embodiments have used CLIP-Seq, TargetScan, and miRNA atlases as tools for identifying targets and designing sequences of the invention.

Cross-linking immunoprecipitation (CLIP) uses UV cross-linking and immunoprecipitation in order to analyze RNA interactions and modifications and has been used with sequencing (dubbed CLIP-Seq) to generate genome-wide RNA interaction maps and for the identification of microRNA targets by decoding microRNA-mRNA and protein-RNA interaction maps in tissue and cell cultures and samples. See Thomson, 2011, Experimental strategies for microRNA target identification, Nucleic Acids Res 39(16):6845-53, incorporated by reference.

TargetScan is a digital tool that predicts biological targets of miRNAs by searching for the presence of conserved 8mer, 7mer, and timer sites that match the seed region of each miRNA. See Lewis, 2005, Conserved Seed Pairing, Often Flanked by Adenosines, Indicates that Thousands of Human Genes are MicroRNA Targets., Cell 120:15-20, incorporated by reference. TargetScan may be used to identify conserved sites, poorly conserved sites, and sites with mismatches in the seed region that are compensated by conserved 3′ pairing, and centered sites. See Friedman, 2009, Most mammalian mRNAs are conserved targets of MicroRNAs, Genome Res 19:92-105 and Shin, 2010, Expanding the microRNA targeting code: functional sites with centered pairing, Mol Cell 38(6):789-802, both incorporated by reference. TargetScan ranks predictions based on the predicted efficacy of targeting as calculated using cumulative weighted context++ scores of the sites. Predictions may also be ranked by their probability of conserved targeting. TargetScanHuman considers matches to human 3′ UTRs and their orthologs, as defined by UCSC whole-genome alignments. Conserved targeting has also been detected within open reading frames (ORFs).

There are atlases of miRNAs. For example, The Human miRNA Tissue Atlas is a catalog of tissue-specific microRNA (miRNA) expression across 62 tissues. See Ludwig, 2016, Distribution of miRNA expression across human tissues, Nucleic Acids Res 44(8):3865-77, incorporated by reference. Another example is the integrated expression atlas of miRNAs and their promoters that was created by deep-sequencing 492 short RNA (sRNA) libraries, with matching Cap Analysis Gene Expression (CAGE) data, from 396 human and 47 mouse RNA samples. See de Rie, 2017, An integrated expression atlas of miRNAs and their promoters in human and mouse, Nat Biotech 35:872-878, incorporated by reference. For that 2017 atlas, promoters were identified for 1,357 human and 804 mouse miRNAs and showed strong sequence conservation between species. It was found that primary and mature miRNA expression levels were correlated, allowing the primary miRNA measurements to be used as a proxy for mature miRNA levels in a total of 1,829 human and 1,029 mouse CAGE libraries. Such tools provide an atlas of miRNA expression and promoters in primary mammalian cells, establishing a foundation for detailed analysis of miRNA expression patterns and transcriptional control regions. Such miRNA atlases may be used to identify targets of the invention.

Nucleic acids of the disclosure were selected by extracting information from CLIP-Seq data and also selected for optimizing a balance of the following requirements: fully block the miRNA seed binding region to thus function as steric blocking oligonucleotides (SBOs), have no (or minimal) off-target hits in human or a model primate (e.g., cynomolgus monkey), have a close match in the model primate (e.g., preferably either exact 20mer or 19/20 nt), balance minimizing overall deltaG, aiming for exact cyno homology or putting mismatches at the ends of the SBO, and avoiding problematic sequence motifs (e.g., high GC content or hairpins) or thermodynamic properties (e.g., extreme Tm). The applied criteria identify target miRNAs to block. Identified miRNAs include miR-491-5p, miR-424-5p, miR-423-5p, miR-423-3p, miR-338-3p, miR-30b-5p, miR-219a-5p, miR-218-5p, miR-154-5p, miR-143-3p, miR-141-3p, and miR-1-3p.

The miR-491-5p target is understood to also function as a tumor suppressor. It may be that blocking the ability of that miRNA to suppress translation of STXBP1 has no relevant significant consequences in its other functions, e.g., suppressing tumors. Similarly, miR-424-5p has been reported to regulate cell proliferation such that interfering with its effects on STXBP1 transcripts suggests itself as a mechanistically reasonable pathway. Literature reports miR 5p and miR-423-3p to be a useful biomarkers or diagnostic indicators and may be implicated in malignant gliomas. It is suspected that miR-338-3p regulates proliferation, apoptosis, and neuronal maturation. The miR-30b-5p is implicated in lipid metabolism and proliferation. The miR-219a-5p is understood to repress EYA2 expression via binding to the 3′-UTR of EYA2. Literature reports that miR-218-5p plays a role in skin and hair follicle development. miR 5p negatively regulates adipose-derived mesenchymal stem cell osteogenic differentiation through the Wnt/PCP pathway by directly targeting Wnt11 3′ UTR. Literature reports that miR-143-3p inhibits osteosarcoma. miR-141-3p suppresses proliferation and promotes apoptosis. Literature reports various roles of miR-1-3p including targeting protein regulator of cytokinesis 1 and inhibiting adenocarcinoma tumorigenesis. The skilled artisan will recognize that such roles for these miRNAs are reported in the literature and may be understood through the use of online libraries of medicine. From the functions of these miRNAs, it may be reasoned that no further adverse effects, which outweigh clinical utility of compositions of the invention, may come from blocking a seed binding site of the miRNA within an STXBP1 transcript. Accordingly, the skilled artisan will recognize that it is mechanistically reasonable to target these targets in STXBP1 transcripts in patient cells, and also that the art-recognized roles of these miRNAs are compatible with such a therapeutic strategy.

Having selected these targets, nucleic acids may be provided (e.g., synthesized) that hybridize to at least a seed binding site of these targets in STXBP1 RNA. Thus, it may be found that a composition of the invention is useful when it includes a nucleic acid with a stretch of at least 5 contiguous bases that are the reverse complement of 5 cognate contiguous bases in STXBP1 RNA to which a miRNA would otherwise bind. Such a nucleic acid is offered as a steric blocking oligonucleotide (SBO) useful for upregulating synthesis of STXBP1 protein by inhibiting the downregulating effect of the miRNA. Thus, the invention provides novel SBOs that block a target miRNA of the disclosure from interfering with STXBP1 protein synthesis. SBOs of the invention preferably include features that promote clinical utility. The disclosed sequences balance minimizing overall deltaG, while possessing homology in a model primate. The sequence preferably locates mismatches at the ends of the SBO, and avoids problematic sequence motifs (e.g., hairpins) or thermodynamic properties. SBOs of the invention based on SEQ ID Nos 1-25 and 74-107 preferably include 2′-O-methoxyethyl-modified ribose sugars (“2′-MOE”) and also preferably include phosphorothioate (PS) inter-base linkages. Optionally, any combination of 2′-O-Methyl and 2′-MOE may be included; any combination of PS and PO backbones may be included. Such features may make the SBOs resistant to hydrolysis or degradation in cells (enzyme- or chemically-catalyzed). Thus, those features may promote a clinically-useful long half-life in vivo for compositions of the invention.

Not only do modifications (e.g., 2′-O-methoxyethyl) at least at one or both of the 5′ and 3′ ends (and preferably throughout) provide increased resistance to nuclease degradation, those modifications may reduce toxicity and provide increased affinity for binding to complimentary RNA. See Vickers, 2001, Fully modified 2′ MOE oligonucleotides redirect polyadenylation, Nucleic Acids Res 29(6):1293-9, incorporated by reference. Compared to standard RNA bases 2′-MOE bases offer increased resistance to nuclease degradation, reduced toxicity, and increased affinity for binding to complimentary RNA.

To increase nuclease resistance, compositions of the invention preferably include phosphorothioate (PS) modifications to the oligo. In a phosphorothioate, a sulfur atom replaces a non-bridging oxygen in the oligo phosphate backbone. PS oligos can provide stability. Phosphorothioate linkages also promote binding to serum proteins, which increases the bioavailability of the ASO and facilitates productive cellular uptake.

Table 1 gives a sequence of bases that may be used in a nucleic acid in a composition of the disclosure.

TABLE 1
Table 1

| | | | | Target |
|---|---|---|---|---|
| Code | Seq | Note | SEQ ID | Description |
| q10 | TGCTCGGGATTTTACCAGTT | m, MOE, | (SEQ ID | miRNA_sites |
| | | PS | NO: 1) | |
| q11 | TGGCTGCTCGGGATTTTACC | m, MOE, | (SEQ ID | miRNA_sites |
| | | PS | NO: 2) | |
| q12 | GATGACTTTGGCTGCTCGGG | m, MOE, | (SEQ ID | miRNA_sites |
| | | PS | NO: 3) | |
| q13 | TTTCTGTGGGGTGAGGATGT | m, MOE, | (SEQ ID | miRNA_sites |
| | | PS | NO: 4) | |
| q14 | AGCAGTTTCTGTGGGGTGAG | m, MOE, | (SEQ ID | miRNA_sites |
| | | PS | NO: 5) | |
| q15 | TTCTTCAGTGTGTCCAGCAG | m, MOE, | (SEQ ID | miRNA_sites |
| | | PS | NO: 6) | |
| q16 | TGGAATGAAGATAGCAGATT | m, MOE, | (SEQ ID | miRNA_sites |
| | | PS | NO: 7) | |
| q17 | TAGGGACTGGAATGAAGATA | m, MOE, | (SEQ ID | miRNA_sites |
| | | PS | NO: 8) | |
| q18 | AGGGGTAGGGACTGGAATGA | m, MOE, | (SEQ ID | miRNA_sites |
| | | PS | NO: 9) | |
| q19 | GAGCAGGCACTGAGGGGTAG | m, MOE, | (SEQ ID | miRNA_sites |
| | | PS | NO: 10) | |
| q20 | CAGGTTATTTGGATGAGAGC | m, MOE, | (SEQ ID | miRNA_sites |
| | | PS | NO: 11) | |
| q21 | GTCACCTCCCAGGTTATTTG | m, MOE, | (SEQ ID | miRNA_sites |
| | | PS | NO: 12) | |
| q22 | TCCTGATTGTCACCTCCCAG | m, MOE, | (SEQ ID | miRNA_sites |
| | | PS | NO: 13) | |
| q23 | GCAGCAGCACAAATGGTGTG | m, MOE, | (SEQ ID | miRNA_sites |
| | | PS | NO: 14) | |
| q24 | AGGTAAACAAGTTTCAAGAC | m, MOE, | (SEQ ID | miRNA_sites |
| | | PS | NO: 15) | |
| q25 | TAAGGTAAACAAGTTTCAAG | m, MOE, | (SEQ ID | miRNA_sites |
| | | PS | NO: 16) | |
| q26 | TAATTTTAAGGTAAACAAGT | m, MOE, | (SEQ ID | miRNA_sites |
| | | PS | NO: 17) | |
| q27 | AACACTGAGATTCTGATAAT | m, MOE, | (SEQ ID | miRNA_sites |
| | | PS | NO: 18) | |
| q28 | AGTACTTTCAAACACTGAGA | m, MOE, | (SEQ ID | miRNA_sites |
| | | PS | NO: 19) | |
| q29 | ATATGTTTGTGCTTCAGTAC | m, MOE, | (SEQ ID | miRNA_sites |
| | | PS | NO: 20) | |
| q30 | GATATATGTTTGTGCTTCAG | m, MOE, | (SEQ ID | miRNA_sites |
| | | PS | NO: 21) | |
| q31 | GTACAGAGATGATATATGTT | m, MOE, | (SEQ ID | miRNA_sites |
| | | PS | NO: 22) | |
| q32 | GGGTAGGGACTGGAATGA | m, MOE, | (SEQ ID | miRNA_sites |
| | | PS | NO: 23) | |
| q33 | GTAGGGACTGGAATGAAGAT | m, MOE, | (SEQ ID | miRNA_sites |
| | | PS | NO: 24) | |
| q34 | GGGTAGGGACTGGAATGAAG | m, MOE, | (SEQ ID | miRNA_sites |
| | | PS | NO: 25) | |
| q35 | CCGCGCUAGGGACCGA | 5, OMe, PO | (SEQ ID | 5prime_hairpin |
| | | | NO: 26) | |
| q36 | CCGCAGCCGCGCUAGGGAC | 5*, OMe, | (SEQ ID | 5prime_hairpin |
| | | PO | NO: 27) | |
| q37 | CGCAGCUCUCCGCCCCGCAG | 5*, OMe, | (SEQ ID | 5prime_hairpin |
| | | PO | NO: 28) | |
| q38 | GUGGGCGCGCUGGGCCAGC | 5*, OMe, | (SEQ ID | 5prime_hairpin |
| | | PO | NO: 29) | |
| q39 | CUCCUCAGGUGGGCGCGC | 5*, OMe, | (SEQ ID | 5prime_hairpin |
| | | PO | NO: 30) | |
| q40 | CCGCCUCCUCAGGUGGGCG | 5*, OMe, | (SEQ ID | 5prime_hairpin |
| | | PO | NO: 31) | |
| q41 | UGCGGACCCCGCCGCCUC | 5, OMe, PO | (SEQ ID | 5prime_hairpin |
| | | | NO: 32) | |
| q42 | GACGCCUGCGGACCCCGC | 5, OMe, PO | (SEQ ID | 5prime_hairpin |
| | | | NO: 33) | |
| q43 | CGACGCCUGCGGACCCC | 5, OMe, PO | (SEQ ID | 5prime_hairpin |
| | | | NO: 34) | |
| q44 | CGCGACGCCUGCGGACC | 5, OMe, PO | (SEQ ID | 5prime_hairpin |
| | | | NO: 35) | |
| q45 | GUCCCGCGACGCCUGCGGAC | 5, OMe, PO | (SEQ ID | 5prime_hairpin |
| | | | NO: 36) | |
| q46 | UCCCGCGACGCCUGCGGA | 5, OMe, PO | (SEQ ID | 5prime_hairpin |
| | | | NO: 37) | |
| q47 | GUCCCGCGACGCCUGCGGA | 5, OMe, PO | (SEQ ID | 5prime_hairpin |
| | | | NO: 38) | |
| q48 | CGUCCCGCGACGCCUGCGGA | 5, OMe, PO | (SEQ ID | 5prime_hairpin |
| | | | NO: 39) | |
| q49 | UCCCGCGACGCCUGCGG | 5, OMe, PO | (SEQ ID | 5prime_hairpin |
| | | | NO: 40) | |
| q50 | CGUCCCGCGACGCCUGCGG | 5, OMe, PO | (SEQ ID | 5prime_hairpin |
| | | | NO: 41) | |
| q51 | UCGUCCCGCGACGCCUGCGG | 5, OMe, PO | (SEQ ID | 5prime_hairpin |
| | | | NO: 42) | |
| q52 | CGUCCCGCGACGCCUGCG | 5, OMe, PO | (SEQ ID | 5prime_hairpin |
| | | | NO: 43) | |
| q53 | UCGUCCCGCGACGCCUG | 5, OMe, PO | (SEQ ID | 5prime_hairpin |
| | | | NO: 44) | |
| q54 | CCGAUCUCCUCGUCCCG | 5*, OMe, | (SEQ ID | 5prime_hairpin |
| | | PO | NO: 45) | |
| q55 | GUCUCCCGGCUCCGAUC | 5*, OMe, | (SEQ ID | 5prime_hairpin |
| | | PO | NO: 46) | |
| q56 | GAGUCUCCCGGCUCCGAU | 5*, OMe, | (SEQ ID | 5prime_hairpin |
| | | PO | NO: 47) | |
| q57 | CGCUGCGCGAGUCUCCCG | 5, OMe, PO | (SEQ ID | 5prime_hairpin |
| | | | NO: 48) | |
| q58 | GGCGCUGCGCGAGUCUCC | 5, OMe, PO | (SEQ ID | 5prime_hairpin |
| | | | NO: 49) | |
| q59 | CCGCGCUAGGGACCGA | 5, OMe, PS | (SEQ ID | 5prime_hairpin |
| | | | NO: 50) | |
| q60 | CCGCAGCCGCGCUAGGGAC | 5*, OMe, | (SEQ ID | 5prime_hairpin |
| | | PS | NO: 51) | |
| q61 | CGCAGCUCUCCGCCCCGCAG | 5*, OMe, | (SEQ ID | 5prime_hairpin |
| | | PS | NO: 52) | |
| q62 | GUGGGCGCGCUGGGCCAGC | 5*, OMe, | (SEQ ID | 5prime_hairpin |
| | | PS | NO: 53) | |
| q63 | CUCCUCAGGUGGGCGCGC | 5*, OMe, | (SEQ ID | 5prime_hairpin |
| | | PS | NO: 54) | |
| q64 | CCGCCUCCUCAGGUGGGCG | 5*, OMe, | (SEQ ID | 5prime_hairpin |
| | | PS | NO: 55) | |
| q65 | UGCGGACCCCGCCGCCUC | 5, OMe, PS | (SEQ ID | 5prime_hairpin |
| | | | NO: 56) | |
| q66 | GACGCCUGCGGACCCCGC | 5, OMe, PS | (SEQ ID | 5prime_hairpin |
| | | | NO: 57) | |
| q67 | CGACGCCUGCGGACCCC | 5, OMe, PS | (SEQ ID | 5prime_hairpin |
| | | | NO: 58) | |
| q68 | CGCGACGCCUGCGGACC | 5, OMe, PS | (SEQ ID | 5prime_hairpin |
| | | | NO: 59) | |
| q69 | GUCCCGCGACGCCUGCGGAC | 5, OMe, PS | (SEQ ID | 5prime_hairpin |
| | | | NO: 60) | |
| q70 | UCCCGCGACGCCUGCGGA | 5, OMe, PS | (SEQ ID | 5prime_hairpin |
| | | | NO: 61) | |
| q71 | GUCCCGCGACGCCUGCGGA | 5, OMe, PS | (SEQ ID | 5prime_hairpin |
| | | | NO: 62) | |
| q72 | CGUCCCGCGACGCCUGCGGA | 5, OMe, PS | (SEQ ID | 5prime_hairpin |
| | | | NO: 63) | |
| q73 | UCCCGCGACGCCUGCGG | 5, OMe, PS | (SEQ ID | 5prime_hairpin |
| | | | NO: 64) | |
| q74 | CGUCCCGCGACGCCUGCGG | 5, OMe, PS | (SEQ ID | 5prime_hairpin |
| | | | NO: 65) | |
| q75 | UCGUCCCGCGACGCCUGCGG | 5, OMe, PS | (SEQ ID | 5prime_hairpin |
| | | | NO: 66) | |
| q76 | CGUCCCGCGACGCCUGCG | 5, OMe, PS | (SEQ ID | 5prime_hairpin |
| | | | NO: 67) | |
| q77 | UCGUCCCGCGACGCCUG | 5, OMe, PS | (SEQ ID | 5prime_hairpin |
| | | | NO: 68) | |
| q78 | CCGAUCUCCUCGUCCCG | 5*, OMe, | (SEQ ID | 5prime_hairpin |
| | | PS | NO: 69) | |
| q79 | GUCUCCCGGCUCCGAUC | 5*, OMe, | (SEQ ID | 5prime_hairpin |
| | | PS | NO: 70) | |
| q80 | GAGUCUCCCGGCUCCGAU | 5*, OMe, | (SEQ ID | 5prime_hairpin |
| | | PS | NO: 71) | |
| q81 | CGCUGCGCGAGUCUCCCG | 5, OMe, PS | (SEQ ID | 5prime_hairpin |
| | | | NO: 72) | |
| q82 | GGCGCUGCGCGAGUCUCC | 5, OMe, PS | (SEQ ID | 5prime_hairpin |
| | | | NO: 73) | |
| q83 | TATTTGGATGAGAGCAGGC | m, MOE, | (SEQ ID | miRNA_sites |
| | | PS | NO: 74) | |
| q84 | TTATTTGGATGAGAGCAGGC | m, MOE, | (SEQ ID | miRNA_sites |
| | | PS | NO: 75) | |
| q85 | GTTATTTGGATGAGAGCAGG | m, MOE, | (SEQ ID | miRNA_sites |
| | | PS | NO: 76) | |
| q86 | GGTTATTTGGATGAGAGCAG | m, MOE, | (SEQ ID | miRNA_sites |
| | | PS | NO: 77) | |
| q87 | GGTAGGGACTGGAATGAAGA | m, MOE, | (SEQ ID | miRNA_sites |
| | T | PS | NO: 78) | |
| q88 | GGGTAGGGACTGGAATGAAG | m, MOE, | (SEQ ID | miRNA_sites |
| | A | PS | NO: 79) | |
| q89 | GGTTATTTGGATGAGAGCAGG | m, MOE, | (SEQ ID | miRNA_sites |
| | | PS | NO: 80) | |
| q90 | GGGTAGGGACTGGAATGAAG | m, MOE, | (SEQ ID | miRNA_sites |
| | AT | PS | NO: 81) | |
| q91 | AGGTTATTTGGATGAGAGCAG | m, MOE, | (SEQ ID | miRNA_sites |
| | G | PS | NO: 82) | |
| q92 | CAGGTTATTTGGATGAGAGCA | m, MOE, | (SEQ ID | miRNA_sites |
| | G | PS | NO: 83) | |
| q93 | TTATTTGGATGAGAGCAGGCA | m, MOE, | (SEQ ID | miRNA_sites |
| | CTG | PS | NO: 84) | |
| q94 | CCATTCATCCTAACAGTCCA | m, MOE, | (SEQ ID | 3Prime_tiling |
| | | PS | NO: 85) | |
| q95 | TCCATACCCAAGCAATGTGC | m, MOE, | (SEQ ID | 3Prime_tiling |
| | | PS | NO: 86) | |
| q96 | TCCAAGAACACAGACATCTC | m, MOE, | (SEQ ID | 3Prime_tiling |
| | | PS | NO: 87) | |
| q97 | GGCACTGTGACTTAGACTGG | m, MOE, | (SEQ ID | 3Prime_tiling |
| | | PS | NO: 88) | |
| q98 | CCTCCAGAACCCATCTGTTC | m, MOE, | (SEQ ID | 3Prime_tiling |
| | | PS | NO: 89) | |
| q99 | GACTAATCTCAGTGCAAGGG | m, MOE, | (SEQ ID | 3Prime_tiling |
| | | PS | NO: 90) | |
| q100 | GGTCCTGAAGCATGAGCACT | m, MOE, | (SEQ ID | 3Prime_tiling |
| | | PS | NO: 91) | |
| q101 | GAACTGAGGCGGGCGGTGGT | m, MOE, | (SEQ ID | 3Prime_tiling |
| | | PS | NO: 92) | |
| q102 | GAGGGCATCACTGAACACGA | m, MOE, | (SEQ ID | 3Prime_tiling |
| | | PS | NO: 93) | |
| q103 | GGCGTCCATTCATCCTAACA | m, MOE, | (SEQ ID | 3Prime_tiling |
| | | PS | NO: 94) | |
| q104 | AGGCGTCCATTCATCCTAAC | m, MOE, | (SEQ ID | 3Prime_tiling |
| | | PS | NO: 95) | |
| q105 | GGGTTATGTACAAGGTCACA | m, MOE, | (SEQ ID | 3Prime_tiling |
| | | PS | NO: 96) | |
| q106 | AGGGTTATGTACAAGGTCAC | m, MOE, | (SEQ ID | 3Prime_tiling |
| | | PS | NO: 97) | |
| q107 | CTACCGTTCCATACCCAAGC | m, MOE, | (SEQ ID | 3Prime_tiling |
| | | PS | NO: 98) | |
| q108 | GGACAGGAACAACCCCAAAC | m, MOE, | (SEQ ID | 3Prime_tiling |
| | | PS | NO: 99) | |
| q109 | CCTCAGTGTGAAATACTCCA | m, MOE, | (SEQ ID | 3Prime_tiling |
| | | PS | NO: 100) | |
| q110 | CGTGCAGACACCCCAGCCTC | m, MOE, | (SEQ ID | 3Prime_tiling |
| | | PS | NO: 101) | |
| q111 | CTTGAGGATGGCGAGACAGC | m, MOE, | (SEQ ID | 3Prime_tiling |
| | | PS | NO: 102) | |
| q112 | GAGGTCAAGGGCGGCGAGGG | m, MOE, | (SEQ ID | 3Prime_tiling |
| | | PS | NO: 103) | |
| q113 | GGAGGTCAAGGGCGGCGAGG | m, MOE, | (SEQ ID | 3Prime_tiling |
| | | PS | NO: 104) | |
| q114 | AACACCACAATGCAGCGAGC | m, MOE, | (SEQ ID | 3Prime_tiling |
| | | PS | NO: 105) | |
| q115 | ATCAGGTCGTATAAGTTGGG | m, MOE, | (SEQ ID | 3Prime_tiling |
| | | PS | NO: 106) | |
| q116 | GGGACAGAACAAGCAGCGGG | m, MOE, | (SEQ ID | 3Prime_tiling |
| | | PS | NO: 107) | |

Table 1 includes a “note” column in which each entry has three parts, separated by commas: mechanism, sugar modification, and inter-base linkages. The first part says either m, 5, or 5*. The second part of each notes says either MOE or OMe. The third part of each note says PS or PO. In the first part, “m” indicates an ASO that operates by blocking binding of an miRNA, “5” indicates an ASO that destabilizes a 5′UTR, and an asterisk (“*”) indicates an ASO that is suspected to mask an uORF. In the second part, “MOE” indicates an ASO in which the sugars are preferred to be substantially or entirely 2′-O-methoxyethyl ribose and “OMe” indicates an ASO in which the sugars are preferred to be substantially or entirely 2′-O-Methyl ribose. In Table 1, SEQ ID NOs: 1-25 and 74-107 use DNA-like characters (shown by the use of T) and SEQ ID NOs: 26-73 use RNA-like characters (shown by the use of U). That usage is consistent with certain industry standards available when ordering synthetic oligonucleotides whereby certain vendors use T when specifying the inclusion of 2′-MOE and U when including 2′-O-Me. In the third part of each note, “PS” indicates an ASO in which the inter-base linkages are preferred to be substantially or entirely phosphorothioate bonds whereas “PO” indicates an ASO in which the inter-base linkages are preferred to be substantially or entirely phosphodiester bonds. For SEQ ID Nos: 26-73, any of the nitrogenous bases may be mC and/or mU. Preferably, mU is used for 2′-MOE bases. “Substantially” includes things that have at least 85% of the recited property. For example, for a 20 base oligo, a person of ordinary skill in the art of molecular biology will recognize that, of the 19 linkages, they could all be PS or they could be substantially all PS (e.g., 18 PS and 1 PO) and the molecule would function essentially similarly.

The sequences listed in Table 1 may be treated as a baseline reference, and a nucleic acid (e.g., a steric blocking oligonucleotide or SBO) in a composition of the invention may be described in comparison to one of the listed sequences. For example, it may be found that mismatches are tolerated, meaning that even where the STXBP1 transcript includes a reverse complement to one of SEQ ID Nos 1-107, the nucleic acid of the invention functions well even when it is less than a 100% match to one of the SEQ ID Nos 1-107. Results suggest that mismatches are best tolerated near the ends of the SBO and also that it is most critical to block a binding region of a miRNA seed sequence, where the seed may be about 7 to 8 bases long, or even as short as 5 or 6. The sequences in Table 1 are 16 to 20 bases long. What may be critical is that a nucleic acid of the invention has a seed region of 5 or 6 or 7 or 8 or 9 contiguous bases that is a 100% match to a corresponding stretch of bases in one of SEQ ID Nos 1-107 and that the nucleic acid of the invention also has at least 50% sequence similarity to one of SEQ ID Nos: 1-3, 7-13, 15-18, and 23-107, at least 60% sequence identity or similarity to one of SEQ ID NOS: 1-3, 7-13, 15-19, and 23-107, at least 70% sequence identity or similarity to one of SEQ ID NOS: 1-3, 7-13, 15-19, and 22-107, at least 75% sequence identity or similarity to one of SEQ ID NOS: 1-3, 6-13, 15-19, and 22-107, at least 80% sequence identity or similarity to one of SEQ ID NOS: 1-3, 6-13, 15-19, and 21-107, at least 85% sequence identity or similarity to one of SEQ ID NOS: 1-3, and 6-107, and/or at least 90% sequence identity or similarity to one of SEQ ID NOS: 1-107.

It is also noted that the certain characters used in Table 1 are presented within the sequences using DNA nomenclature (e.g., using the letters A, T, C, and G) and are silent as to ribose sugar composition or inter-base linkages. It may be found that the nucleic acid of the invention is most useful with RNA bases (e.g., uracil for the nitrogenous base where T is shown) and also or alternatively with modified ribose sugars (e.g., 2′-MOE). In fact, it may be found that the letter T in a listed sequence can be present in a nucleic acid of the invention as the nucleobase thymine or uracil and/or even that those bases can be mixed or intermingled along the SBO. In some embodiments, a nucleic acid of the invention includes 2′-MOE “methylated” U (5-methyluridine), which in essence is a 2′-MOE-T.

In particular, 2′-MOE bases may use 5-methyl cytosine and 5-methyl uridine. It may be preferable to use 5-methyl cytosine to avoid non methylated CpG. It may be found that avoiding non-methylated CpG decreases or avoids inflammatory potential. Additionally, 5-methyl cytosine 2′-MOE bases may be found to be aligned with clinically validated chemistry and may optionally be preferred for such a reason. The 5-methyl-U (T) bases may be used with 2′-MOE chemistry for ease of manufacturing and commercial availability. For sequences using 2′-OMe chemistry, ease of manufacturing and/or commercial availability may favor not using 5-methyl C and/or 5-methly-U (T).

In certain embodiments, a majority or all of the bases represented by the letter T have the nucleobase uracil. In preferred embodiments, a majority or all of the bases represented by the letter T have a 2′-MOE-T. Any, most, or all of the linkages may be phosphorothioate. A preferred embodiment uses all phosphorothioate linkages for SEQ ID Nos 1-25, 50-107. Preferred embodiments use phosphodiester for SEQ ID Nos: 26-49.

In fact, one first embodiment provides a composition with a nucleic acid for use as an ASO to promote STXBP1 expression. The nucleic acid has a base sequence with an at least 88% match to one of SEQ ID Nos 1-107 (i.e., no greater than two mismatches). All of the nitrogenous bases are A, T, U, C, or G, optionally with mC. All of the sugars are 2′-MOE for SEQ ID Nos: 1-25 and 74-107 and all of the linkages are PS for SEQ ID Nos 1-25 and 50-107. Sugars are 2′-OMe for SEQ ID Nos: 26-73. These first embodiments are attractive for ease of manufacture. Such ASOs can be synthesized on standard benchtop RNA-synthesis systems and/or ordered from commercial vendors.

Stated more generally, preferably at least about 6 to 12 contiguous bases in the nucleic acid have at least 90% sequence identity to a corresponding 12 contiguous bases in one of SEQ ID Nos: 1-107 (to stably bind to target). Preferably at least about 6 to 12 contiguous bases in the nucleic acid have at least 90% sequence identity to a corresponding 12 contiguous bases in one of SEQ ID Nos: 1-25 (e.g., to block the seed region of the implicated miRNA).

Preferably: a majority of the bases of the nucleic acid have a 2′-O-methoxyethyl-modified ribose (in SEQ ID Nos 1-25 and 74-107), a majority of inter-base linkages in the nucleic acid are phosphorothioate bonds (in SEQ ID Nos 1-25 and 50-107), and nitrogenous bases are A, T, U, C, G, or mC. As suggested above, SEQ ID Nos: 1-25 and 74-84 in Table 1 are specifically designed to block specific miRNAs from downregulating STXBP1 transcripts; SEQ ID Nos: 26-73 are designed to destabilize 5′UTR hairpins; and SEQ ID NOS: 85-107 are designed to target 3′ regulatory regions.

In miR-423-3p embodiments, the nucleic acid hybridizes to a binding site of, and blocks binding to an STXBP1 transcript of, miR-423-3p. For example, the nucleic acid may have at least 50% or 75% or 83% or 90% or 95% or 100% sequence similarity to one of SEQ ID Nos: 1, 2 and 3. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxy-ethyl-modified ribose, a majority of inter-base linkages in the nucleic acid are phosphorothioate bonds, and/or any nitrogenous bases are RNA chemistry (e.g., the letter T indicates 5-methyl uracil). It may be preferably that the letter C represent 5′-methyl cytosine. The nucleic acid may have a base sequence with an at least 88% match to the indicated sequence (i.e., no greater than two mismatches). All of the sugars may be 2′-MOE and all of the linkages may be PS.

In miR-491-5p embodiments, the nucleic acid hybridizes to a binding site of, and blocks binding to an STXBP1 transcript of, miR-491-5p. For example, the nucleic acid may have at least 90% or 95% or 100% sequence similarity to one of SEQ ID Nos: 4 and 5. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′ methoxy-ethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-338-3p embodiments, the nucleic acid hybridizes to a binding site of, and blocks binding to an STXBP1 transcript of, miR-338-3p. For example, the nucleic acid may have at least 75% or 85% or 90% or 95% or 100% sequence similarity to SEQ ID NO: 6. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-1-3p embodiments, the nucleic acid hybridizes to a binding site of, and blocks binding to an STXBP1 transcript of, miR-1-3p. For example, the nucleic acid may have at least 50% or 75% or 85% or 90% or 95% or 100% sequence similarity to one of SEQ ID Nos: 7, 8, 9, 23, 24, and 25. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C). The nucleic acid may have a base sequence with an at least 90% match to the indicated sequence (i.e., no greater than two mismatches). All of the sugars may be 2′-MOE and/or 2′OMe and all of the linkages may be PS and/or PO.

In miR-423-5p embodiments, the nucleic acid hybridizes to a binding site of, and blocks binding to an STXBP1 transcript of, miR-423-5p. For example, the nucleic acid may have at least 50% or 75% or 85% or 90% or 95% or 100% sequence similarity to SEQ ID NO: 10. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-154-5p embodiments, the nucleic acid hybridizes to a binding site of, and blocks binding to an STXBP1 transcript of, miR-154-5p. For example, the nucleic acid may have at least 50% or 75% or 85% or 90% or 95% or 100% sequence similarity to one of SEQ ID Nos: 11 and 12. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-219a-5p embodiments, the nucleic acid hybridizes to a binding site of, and blocks binding to an STXBP1 transcript of, miR-219a-5p. For example, the nucleic acid may have at least 50% or 75% or 85% or 90% or 95% or 100% sequence similarity to SEQ ID NO: 13. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-424-5p embodiments, the nucleic acid hybridizes to a binding site of, and blocks binding to an STXBP1 transcript of, miR-424-5p. For example, the nucleic acid may have at least 85% or 90% or 95% or 100% sequence similarity to SEQ ID NO: 14. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-30b-5p embodiments, the nucleic acid hybridizes to a binding site of, and blocks binding to an STXBP1 transcript of, miR-30b-5p. For example, the nucleic acid may have at least 50% or 75% or 85% or 90% or 95% or 100% sequence similarity to one of SEQ ID Nos: 15, 16, and 17. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-141-3p embodiments, the nucleic acid hybridizes to a binding site of, and blocks binding to an STXBP1 transcript of, miR-141-3p. For example, the nucleic acid may have at least 50% or 75% or 85% or 90% or 95% or 100% sequence similarity to SEQ ID NO: 18 or at least 60% or 75% or 85% or 90% or 95% or 100% sequence similarity to SEQ ID NO: 19. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 90% match to the indicated sequence (i.e., no greater than two mismatches). All of the sugars may be 2′-MOE and all of the linkages may be PS.

In miR-218-5p embodiments, the nucleic acid hybridizes to a binding site of, and blocks binding to an STXBP1 transcript of, miR-218-5p. For example, the nucleic acid may have at least 85% or 90% or 95% or 100% sequence similarity to SEQ ID NO: 20 or at least 80% or 85% or 90% or 95% or 100% sequence similarity to SEQ ID NO: 21. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 88% match to the indicated sequence (i.e., no greater than two mismatches). All of the sugars may be 2′-MOE and all of the linkages may be PS.

In miR-143-3p embodiments, the nucleic acid hybridizes to a binding site of, and blocks binding to an STXBP1 transcript of, miR-143-3p. For example, the nucleic acid may have at least 70% or 75% or 83% or 90% or 95% or 100% sequence similarity to SEQ ID NO: 22. Preferably at least about 6 to 12 contiguous bases in the nucleic acid have at least 90% or 100% sequence identity to a corresponding segment of contiguous bases in the indicated sequence (to block the seed region of the implicated miRNA) and preferably: a majority of the bases of the nucleic acid have a 2′-O-methoxyethyl-modified ribose, a majority of inter-base linkages in the nucleic acid are phosphorothioate bonds, and/or any nitrogenous bases use 5-methyl uracil (T) for T and 5-methyl cytosine for C. The nucleic acid may have a base sequence with an at least 88% match to the indicated sequence (i.e., no greater than two mismatches). All of the sugars may be 2′-MOE and all of the linkages may be PS.

Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention may be man-made, i.e., chemically synthesized, and is typically purified or isolated. The oligonucleotide of the invention may comprise one or more modified nucleosides or nucleotides, such as 2′ sugar modified nucleosides.

The modified nucleotides may be independently selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-O-methoxyethyl modified nucleotide, a 2′-O-alkyl-modified nucleotide, a morpholino nucleotide, a phosphoramidate, a non-natural base comprising nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2′-O-(N-methylacetamide) modified nucleotide, and combinations thereof.

The nitrogenous bases of the ASO may be naturally occurring nucleobases such as adenine, guanine, cytosine, thymidine, uracil, xanthine and hypoxanthine, as well as non-naturally occurring variants, such as substituted purine or substituted pyrimidine, such as nucleobases selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, 5-methyl cytosine LNA nucleosides may be used.

An oligonucleotide of the disclosure is capable of up-regulating the expression of STXBP1.

An oligonucleotide of the disclosure may comprise one or more nucleosides which have a modified sugar moiety, i.e., a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those where the ribose ring structure is modified, e.g., by replacement with a hexose ring (HNA), or a bicyclic ring, which typically have a bridge between the C2 and C4 carbons on the ribose ring (LNA), or an unlinked ribose ring which typically lacks a bond between the C2 and C3 carbons (e.g., UNA). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

The oligonucleotide may include one or more Locked Nucleic Acid (LNA) bases. An LNA may include a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex. Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, and WO 2008/150729, all incorporated by reference.

Pharmaceutically acceptable salts of oligonucleotides of the disclosure include those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, a sulfonic acid, or salicylic acid. In addition, those salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins.

An ASO may comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, 2′-MOE units, arabino nucleic acid (ANA) units, 2′-fluoro-ANA units, or combinations thereof.

Conjugation of the oligonucleotide to one or more non-nucleotide moieties may improve the pharmacology of the oligonucleotide, e.g., by affecting the activity, cellular distribution, cellular uptake or stability of the oligonucleotide. In some embodiments the conjugate moiety can modify or enhance the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. The conjugate may also serve to reduce activity of the oligonucleotide in non-target cell types, tissues, or organs, e.g., off target activity or activity in non-target cell types, tissues, or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids) or combinations thereof. In some embodiments, an ASO of the invention is conjugated to an antibody. For example, antibodies may be conjugated to ASOs to promote or facilitate delivery of the ASOs.

A composition of the disclosure may be provided in pharmaceutical compositions that include any of the aforementioned nucleic acids or salts thereof and a pharmaceutically acceptable diluent, carrier, salt and/or adjuvant. A pharmaceutically acceptable diluent includes ACSF artificial cerebrospinal fluid and pharmaceutically acceptable salts include, but are not limited to, sodium and potassium salts. In some embodiments the pharmaceutically acceptable diluent is sterile phosphate buffered saline or sterile sodium carbonate buffer. In some preferred embodiments, diluents for clinical application include Elliotts B solution and/or ACSF artificial cerebrospinal fluid.

In some embodiments the oligonucleotide of the invention is in the form of a solution in the pharmaceutically acceptable diluent, for example dissolved in saline, PBS, or sodium carbonate buffer. The oligonucleotide may be pre-formulated in the solution or in some embodiments may be in the form of a dry powder (e.g., a lyophilized powder) which may be dissolved in the pharmaceutically acceptable diluent prior to administration. Suitably, for example the oligonucleotide may be dissolved in a concentration of 0.1-100 mg/mL, such as 1-10 mg/mL.

The invention provides methods of treating an early onset epileptic encephalopathy by delivering to a patient in need thereof any of the compositions of the disclosure. The delivered composition may include one or any combination of the disclosed nucleic acids. To say “one nucleic acid” does not mean a single molecule but rather a composition that includes, e.g., millions of, chemically identical copies of the molecule. It may be preferable to deliver two or more of the nucleic acids in combination or at different times to the same patient. Preferably, the composition is delivered across the blood-brain barrier. The nucleic acid may be delivered using a vector that promotes crossing the blood brain barrier including e.g., certain viral vectors (e.g., adeno-associated viral vectors) that serve such purpose. The nucleic acid may be packaged with a particle such as for example a viral vector, a vesicle, or a lipid nanoparticle. The delivery particle may, in-turn, be decorated with ligands that promote delivery, such as, for example, antibodies known to promote receptor-mediated transcytosis across the blood-brain barrier. For a review, see Stanimirovic, 2018, Emerging technologies for delivery of biotherapeutics and gene therapy across the blood-brain barrier, BioDrugs 32(6):547-559, incorporated by reference. The composition may be delivered by e.g., systemic or intrathecal injection. The nucleic acid is delivered to increase expression of STXBP1 in the patient. Methods may include selecting the patient by identifying that the patient carries a heterozygous loss-of-function mutation in a STXBP1 gene.

### EXAMPLES

### Example 1: STXBP1 ASO Program

In miRNA embodiments, 25 miRNA-blocking ASOs were designed as described herein. Those were all 20-mer ASOs with 2′-MOE chemistry and PS backbones. Those are represented by SEQ ID Nos: 1-25.

In UTR embodiments, 48 5′ UTR hairpin-blocking ASOs were designed and made. Those had various lengths ranging from 16-mers to 20-mers. All bases are 2′-O-Methyl. 24 ASOs have PO linkages. 24 ASOs have PS linkages. Initial screening was done at 200 nM, 100 nM, and/or 50 nM.

The PO UTR ones with the PO backbone are represented by SEQ ID NOs: 26-49.

The PS UTR ones with the PS backbone are represented by SEQ ID NOs: 50-73.

Screening was conducted on Fibroblasts, iPSC-derived NGN2 neurons, and/or SH-SY5Y neuroblastoma cells. At least 3 protocols were employed: (i) fibroblast and NGN2 neuron experiments; (ii) SH-SY5Y neuroblastoma experiments, and (iii) NGN2 neuron/Glia coculture experiments. For (i) fibroblast and NGN2 neuron experiments, cells were plated at DIV0 (day in vitro 0). At DIV03, ASO Treatment (Tx). At DIV10 (7 days of ASO treatment) harvest. At DIV13 harvest (was a 10-day ASO treatment). In (ii) SH-SY5Y neuroblastoma experiments, Tx at DIV01; 48 hr harvest at DIV03, and 96 hr harvest at DIV05. For (iii) NGN2 neuron/Glia co-culture experiments, Treatment at DIV20, 4 day post-ASO treatment harvest at DIV24, and 10 day post-ASO treatment harvest at DIV30.

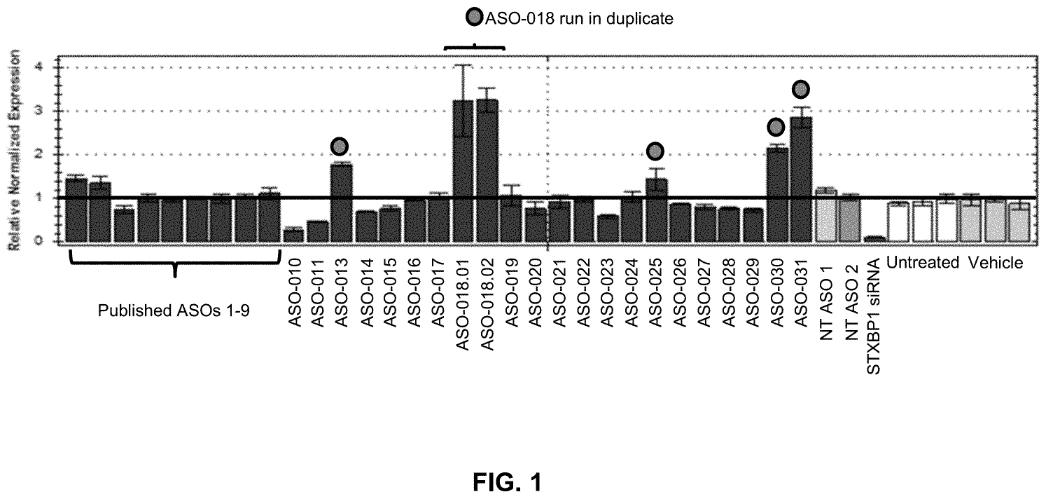

Throughout the examples and in the corresponding figure, an ASO number is given by the code number in Table 1. Thus, for example, ASO-018 referenced in FIG. 1 is also code q18 in Table 1 and represented by SEQ ID NO: 9. Or, similarly, ASO-034 is also code q34 and represented by SEQ ID NO: 25.

FIG. 1 shows results from screening of 21 STXBP1 miRNA-blocking ASOs: control fibroblast (7 days post-treatment). A circle drawn above a bar indicates an ASO selected for dose-response test. Human fibroblasts were plated at 8 k per well and cultured for a total of 10 days. STXBP1 ASOs were transfected into fibroblasts at 200 nM on day 3 using RNAi Max. Cells were collected 7 days post-treatment on day 10 and qPCR was used to assess STXBP1 transcript expression. Each bar represents 2 technical replicates from a single biological replicate. STXBP1 expression is normalized to Actin.

Experimental Parameters:

qPCR; Cell Type: Control Fibroblasts (DIV10); Transfection: RNAi Max 0.5 uL; Treatment: ASOs at 200 nM; qPCR: 7 days after ASO treatment; Target Gene: STXBP1 (Hs00162430_m1); and Control Gene: hActin (Hs999999903_m1).

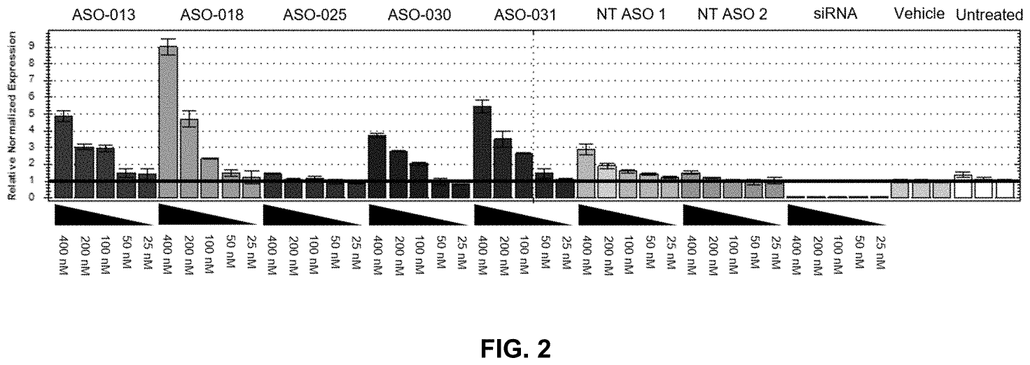

FIG. 2 shows dose response of 5 STXBP1 miRNA-blocking ASOs in Fibroblasts. Human fibroblasts were plated at 8 k per well and cultured for a total of 10 days. STXBP1 ASOs were transfected into fibroblasts at either 25, 50, 100, 200, or 400 nM on day 3 using RNAi Max. Cells were collected 7 days post-treatment on day 10 and qPCR was used to assess STXBP1 transcript expression. Each bar represents 2 technical replicates from a single biological replicate. STXBP1 expression is normalized to Actin.

A key result is that STXBP1 expression level exhibits a dose-dependent response to treatment with compositions of the invention.

Experimental Parameters:

qPCR; Cell Type: Control Fibroblast (DIV10); Transfection: RNAi Max 0.5 uL; Treatment: ASOs at 400 nM, 200 nM, 100 nM, 50 nM and 25 nM (DIV3); qPCR: 7 days after ASO treatment; Target Gene: STXBP1 (Hs00162430_m1); Control Gene: hActin (Hs999999903_m1).

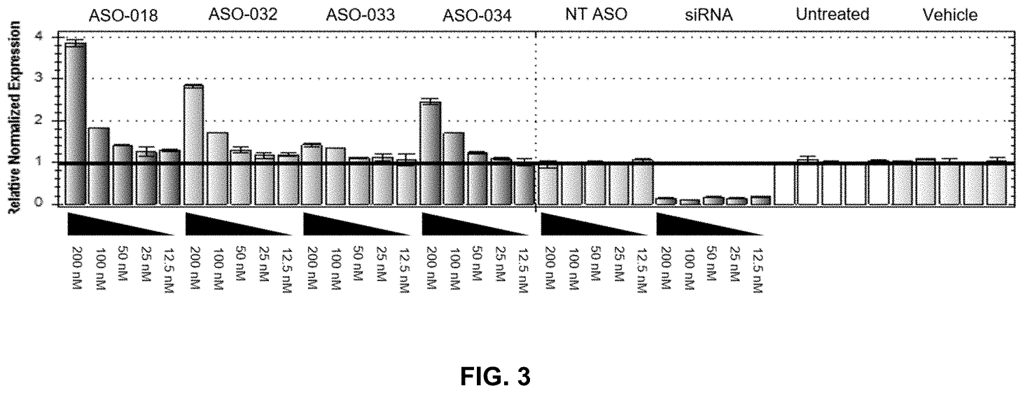

FIG. 3 shows dose response of ASO-018 and 3 Additional ASOs Derived from ASO-018 with Modified Sequences in Fibroblasts. Human fibroblasts were plated at 5 k per well and cultured for a total of 10 days. STXBP1 ASOs were transfected into fibroblasts at either 12.5, 25, 50, 100, or 200 nM on day 3 using RNAi Max. Cells were collected 7 days post-treatment on day 10 and qPCR was used to assess STXBP1 transcript expression. Each bar represents 2 technical replicates from a single biological replicate. STXBP1 expression is normalized to GAPDH.

A key result is that STXBP1 expression level exhibits a dose-dependent response to treatment with compositions of the invention.

Experimental Parameters:

qPCR; Cell Type: Human Fibroblasts (DIV10); Transfection: LipoRNAiMax 0.5 uL; Treatment: ASOs at 200 nM, 100 nM, 50 nM, 25 nM, and 12.5 nM (DIV3); qPCR: 7 days after ASO treatment; Target Gene: STXBP1 (Hs00162430_m1); and Control Gene: GAPDH (Hs02758991_g1).

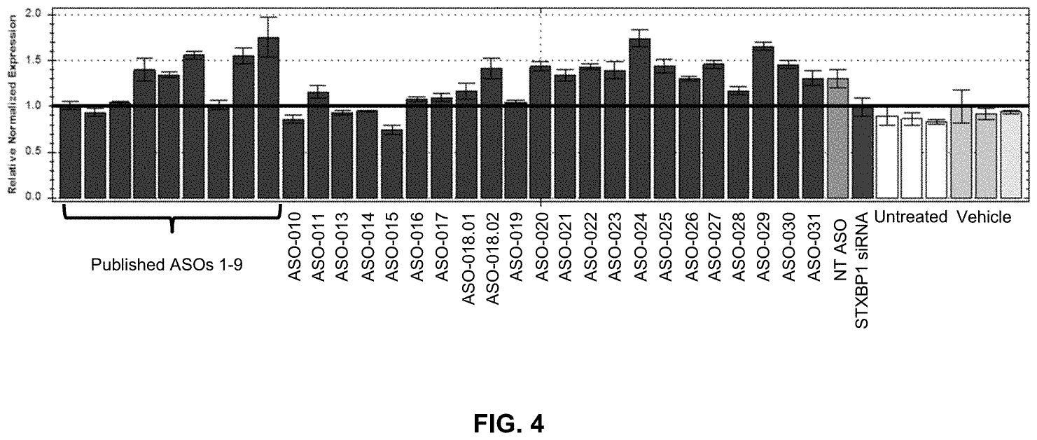

FIG. 4 shows results from screening of 21 STXBP1 miRNA-blocking ASOs: iPSC derived NGN2 neurons (7 days post-treatment). Human iPSC-derived NGN2 excitatory neurons were plated at 80 k per well and cultured for a total of 10 days. STXBP1 ASOs were transfected into neurons at a single concentration of 200 nM on day 3. Cells were collected 7 days post-treatment on day 10 and qPCR was used to assess STXBP1 transcript expression. Each bar represents 2 technical replicates from a single biological replicate. STXBP1 expression is normalized to Actin.

The ASOs with codes q11 and q15 through q31, which are SEQ ID Nos: 2 and 6 through 22 show the strongest effects on STXBP1 expression.

Experimental Parameters:

qPCR; Cell Type: NGN2 neurons (DIV10); Transfection: Endoporter PEG 0.6 uL; Treatment: ASOs at 200 nM; qPCR: 7 days after ASO treatment; Target Gene: STXBP1 (Hs00162430_m1); and Control Gene: hActin (Hs999999903_m1).

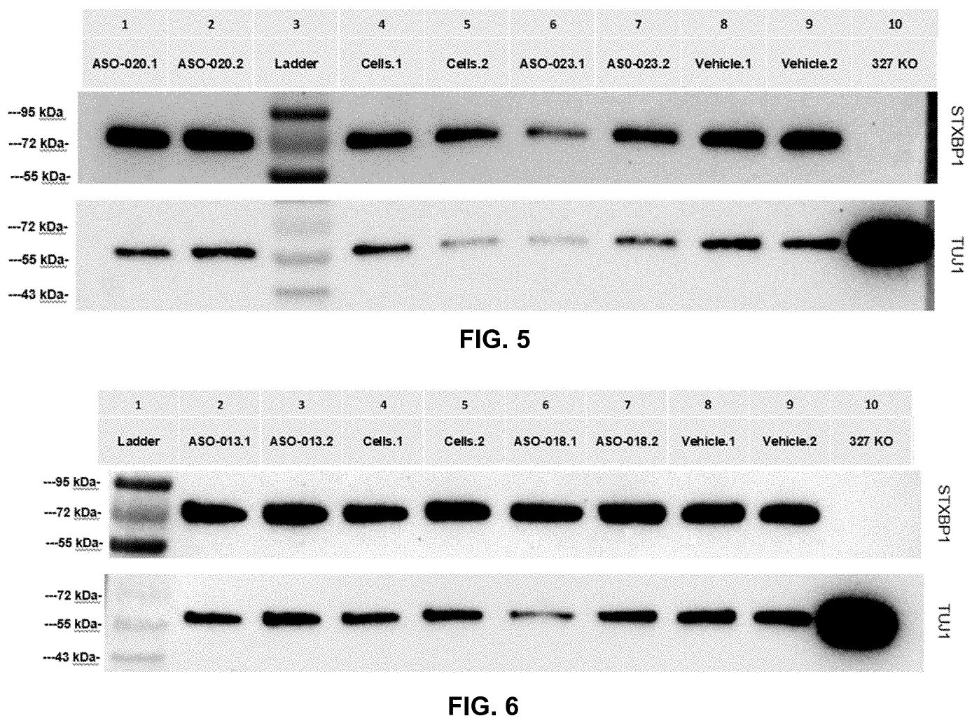

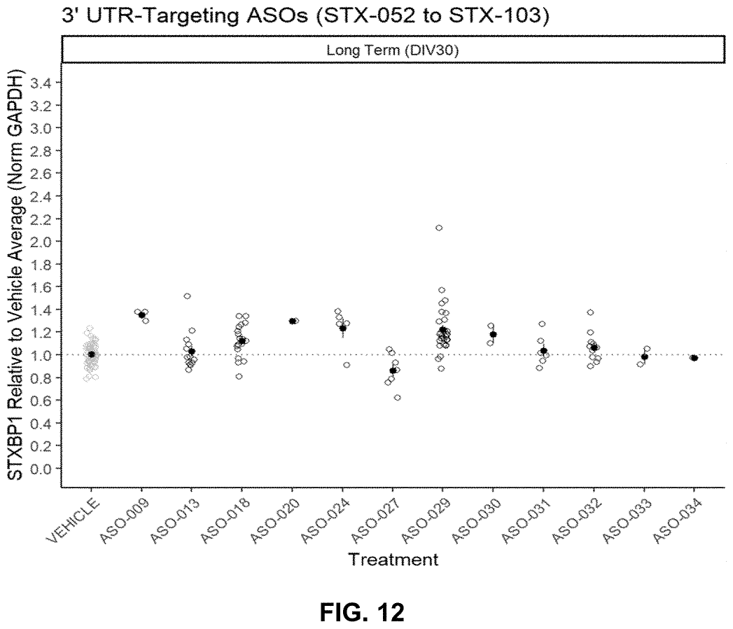

FIGS. 5-8 show results for screening 8 exemplary STXBP1 miRNA-blocking ASOs in SH-SY5Y Neuroblastoma cells 48 hours post-treatment. SH-SY5Y neuroblastoma cells were plated at 45 k per well and cultured for a total of 3 days. STXBP1 ASOs were transfected into cells at a single concentration of 100 nM on day 1 using RNAi Max. Cells were collected 48-hours post-treatment on day 3 and Western Blot was used to assess STXBP1 expression. TUJ1 was used as a marker for protein load. STXBP1 protein levels were then normalized within sample to TUJ1 and quantified as shown in FIG. 9. “.1” and “.2” indicate technical replicates from the same biological replicate.

FIG. 5 shows results from screening of 2 STXBP1 miRNA-blocking ASOs (q20 & q23): SH-SY5Y Neuroblastoma Cells (48 hours post-treatment).

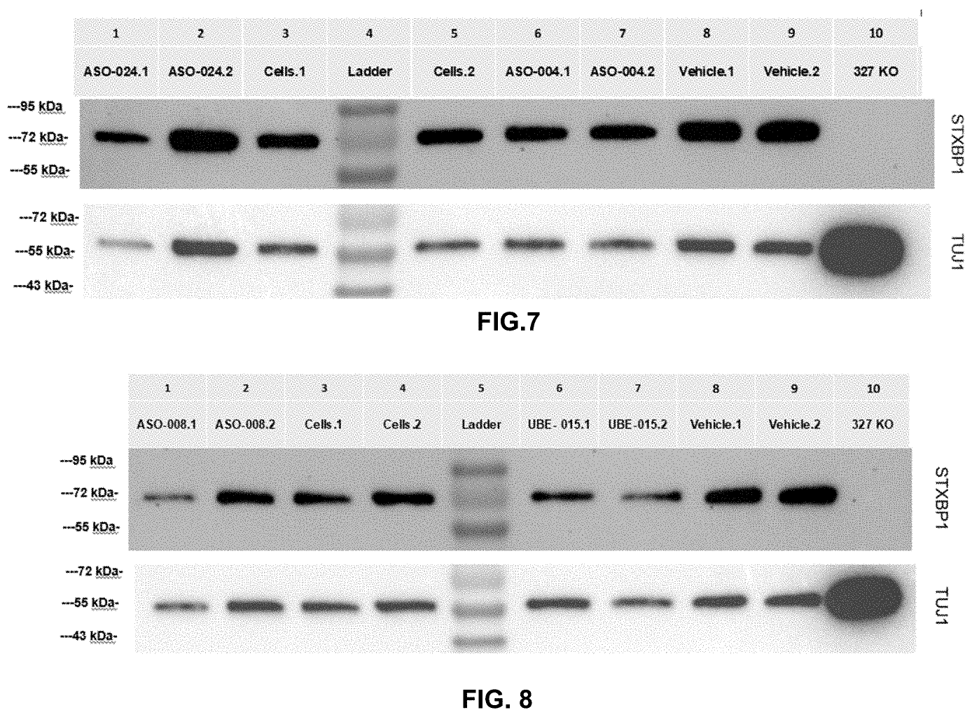

FIG. 6 shows results from screening of 2 STXBP1 miRNA-blocking ASOs (q13 & q18): SH-SY5Y Neuroblastoma Cells (48 hours post-treatment).

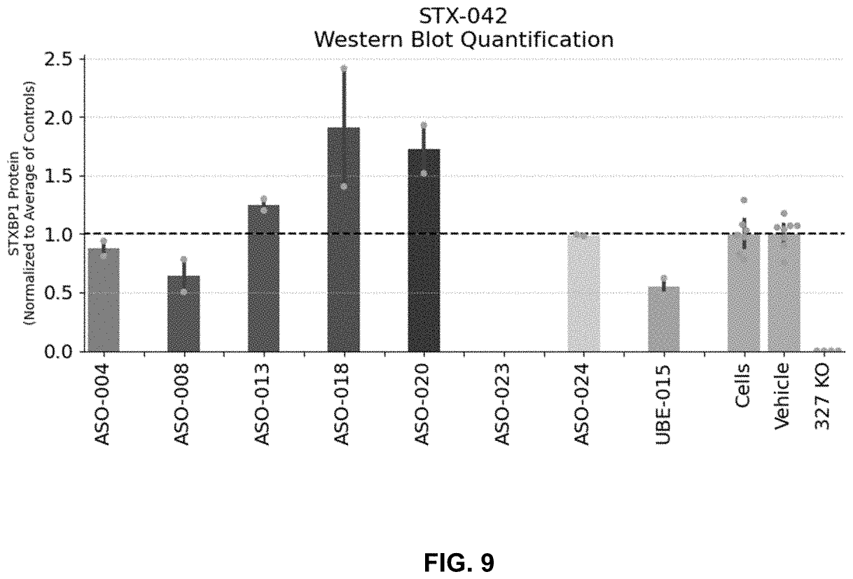

FIG. 7 shows results from screening of 2 STXBP1 miRNA-blocking ASOs (q24 & q4): SH-SY5Y Neuroblastoma Cells (48 hours post-treatment).

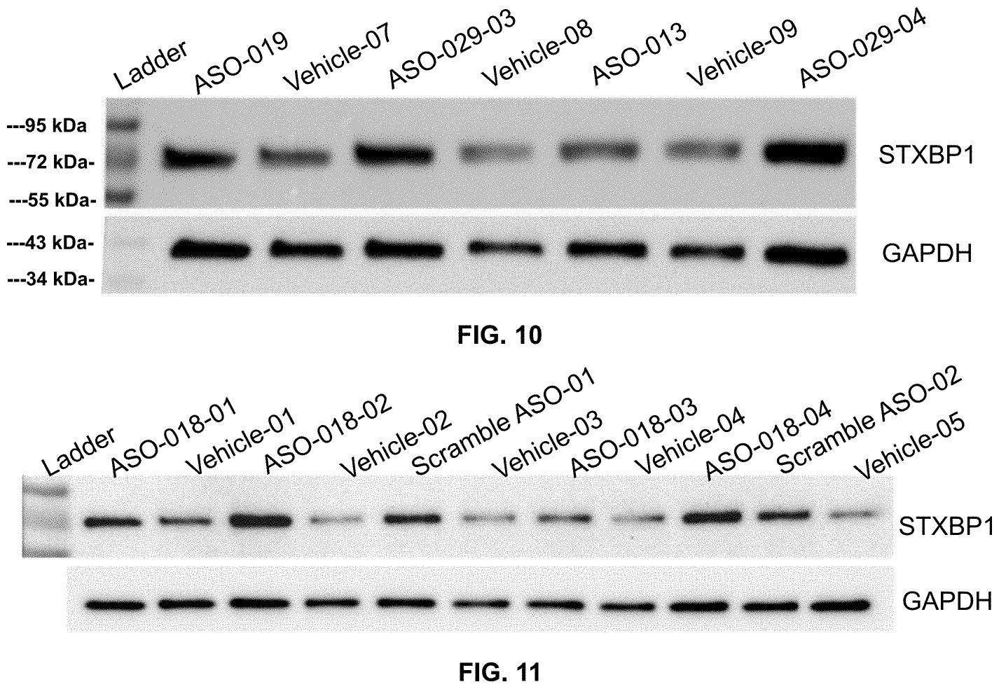

FIG. 8 shows results from screening a STXBP1 miRNA-blocking ASO (q8): SH-SY5Y Neuroblastoma Cells (48 hours post-treatment).

Experimental Parameters:

For screening q20, q23, q13, q18, q24, q4, and q8 with SH-SY5Y neuroblastoma cells: Cell Type: SH-SY5Y Cells (DIV3); Transfection: RNAi Max (0.5 uL) (DIV1); Treatment: ASOs at 100 nM; Protein Harvest: 48 hrs after ASO treatment; Primary Target antibody: Rabbit (Rb) anti-Munc18-1 (Cell Signaling); and Control Target antibody: Mouse (Ms) anti-Beta III Tubulin (TUJ1).

FIG. 9 shows results from Screening of 8 STXBP1 miRNA-blocking ASOs: SH-SY5Y Neuroblastoma Cells (48 hours post-treatment). SH-SY5Y neuroblastoma cells were plated at 45 k per well and cultured for a total of 3 days. STXBP1 ASOs were transfected into cells at a single concentration of 100 nM on day 1 using RNAi Max. Cells were collected 48-hours post-treatment on day 3 and Western Blot was used to assess STXBP1 expression—Western Blots presented in FIGS. 5-8. TUJ1 was used as a marker for protein load. STXBP1 protein levels were then normalized within sample to TUJ1 to generate relative protein expression. The relative STXBP1 protein expression for each condition was then normalized to the cells condition to generate the relative normalized expression.

Experimental Parameters:

Cell Type: SH-SY5Y Cells (DIV3); Transfection: RNAi Max (0.5 uL) (DIV1); Treatment: ASOs at 100 nM; Protein Harvest: 48 hrs after ASO treatment; Primary Target antibody: Rb anti-Munc18-1 (Cell Signaling); and Control Target antibody: Ms anti-Beta III Tubulin (TUJ1).

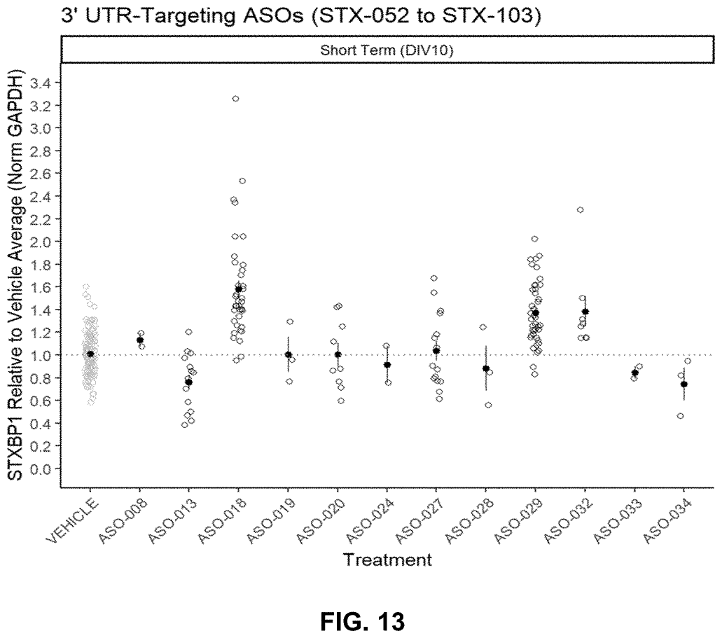

FIGS. 10-11 show images of gels that provide the results of an example screening of STXBP1 protein boosting with miRNA-blocking ASOs in human iPSC-derived NGN2 neurons. Human iPSC-derived NGN2 excitatory neurons were plated at a density between 20 and 50 k per well and cultured for a total of 10 days. STXBP1 ASOs were transfected into neurons at a single concentration of 100 nM on day 4. Cells were collected 6 days post-treatment on day 10 and Western Blot was used to assess STXBP1 expression. GAPDH was used as a control marker/protein for protein loading. “−01”,“−02”, “03”, etc. indicate biological replicates.

FIG. 10 shows an image of a western blot gel providing results for screening 6-days post-treatment of 4 STXBP1 miRNA-blocking ASOs in human iPSC-derived NGN2 neurons. The ASOs tested included ASO-19 (which includes the sequence of SEQ ID NO: 10), ASO-029 (which includes the sequence of SEQ ID NO: 20), and ASO-013 (which includes the sequence of SEQ ID NO: 4).

FIG. 11 shows an image of a western blot gel providing results for screening 6-days post-treatment of 6 STXBP1 miRNA-blocking ASOs in human iPSC-derived NGN2 neurons. The ASOs tested included ASO-18 (which includes the sequence of SEQ ID NO: 9).

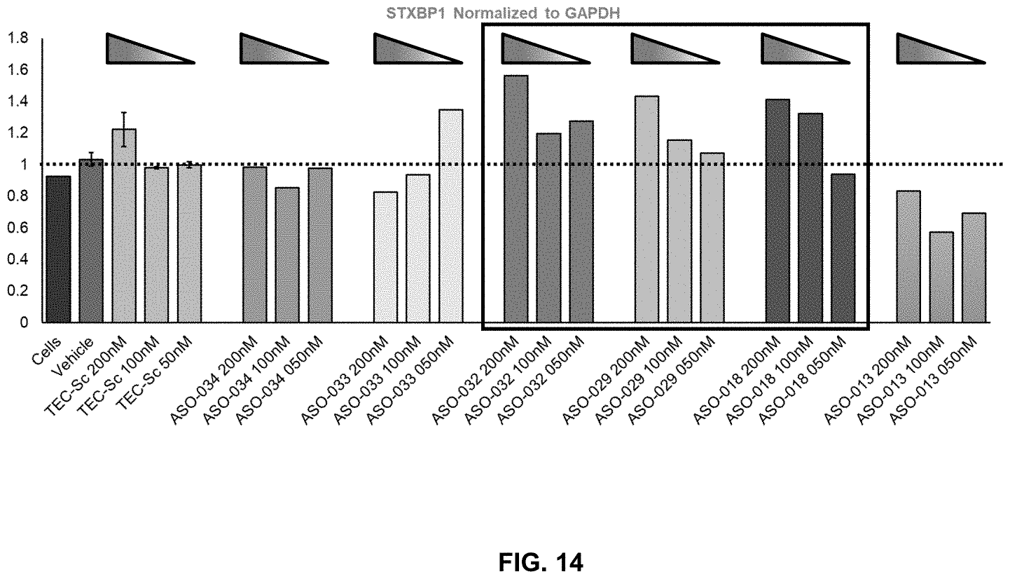

FIGS. 12-13 provide a summary of screening for boosting of STXBP1 protein across several 3′ miRNA-targeting ASOs in human iPSC-derived NGN2 neurons at 2 timepoints in culture. Human iPSC-derived NGN2 excitatory neurons were plated at a density between 20 and 50 k per well and cultured for a total of 30 days (FIG. 12) or 10 days (FIG. 13). STXBP1 ASOs were transfected into neurons at a single concentration of 100 or 200 nM on either day 20 (FIG. 12) or day 4 (FIG. 13). Cells were collected either 10 days (FIG. 12) or 6 days (FIG. 13) post-treatment and Western Blot was used to assess STXBP1 expression. GAPDH was used as a control marker/protein for protein loading. Example Western Blots are presented in FIGS. 10-11. GAPDH was used as a marker for protein load. STXBP1 protein levels were normalized within sample to GAPDH to generate relative protein expression. The relative STXBP1 protein expression for each condition was then normalized to the vehicle condition to generate the relative normalized expression. Each dot per ASO represents an independent biological replicate.

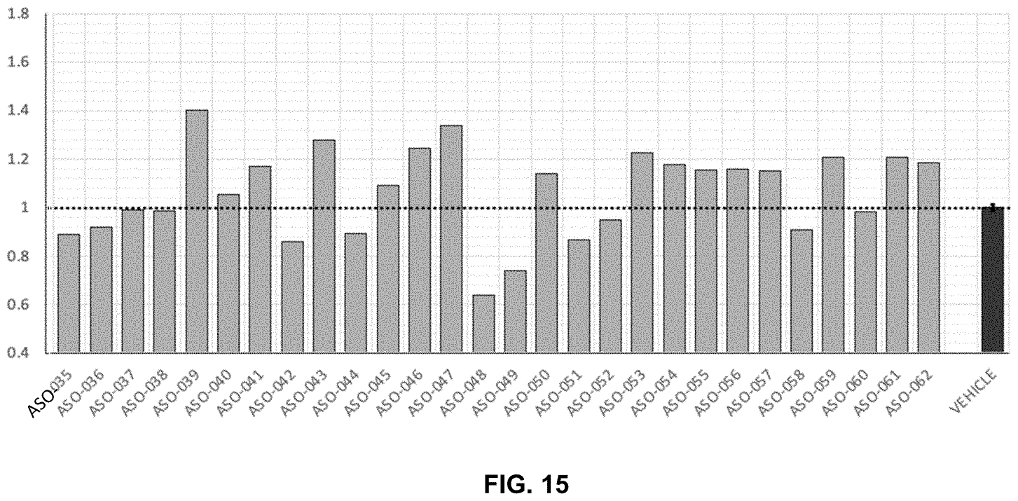

FIG. 14 provides results showing that STXBP1 ASO hits modulate STXBP1 protein in dose-response in human iPSC-derived NGN2 neurons. Human iPSC-derived NGN2 excitatory neurons were plated at a density between 20 and 50 k per well and cultured for a total of 10 days. STXBP1 ASOs were transfected into neurons at a concentration of 50, 100, or 200 nM on day 4. Cells were collected 6 days post-treatment and Western Blot was used to assess STXBP1 expression. GAPDH was used as a control marker/protein for protein loading. STXBP1 protein levels were normalized within sample to GAPDH to generate relative protein expression. The relative STXBP1 protein expression for each condition was then normalized to the vehicle condition to generate the relative normalized expression. Hit ASOs from a single concentration screen are marked in the box.

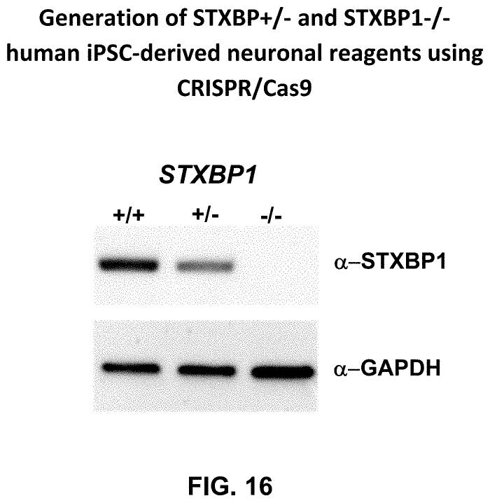

FIG. 15 shows quantification of 5′ STXBP1 ASOs screened in human iPSC-derived neurons with Western Blotting revealing STXBP1 protein boosting for several ASOs of the invention. Human iPSC-derived NGN2 excitatory neurons were plated at a density between 20 and 50 k per well and cultured for a total of 30 days. STXBP1 ASOs were transfected into neurons at a concentration of 100 nM on day 20. Cells were collected 10 days post-treatment and Western Blot was used to assess STXBP1 expression. GAPDH was used as a control marker/protein for protein loading. STXBP1 protein levels were normalized within sample to GAPDH to generate relative protein expression. The relative STXBP1 protein expression for each condition was then normalized to the vehicle condition (right-most bar) to generate the relative normalized expression. All rounds are from iPSC-derived NGN2 neurons treated with ASO (100 nM) at ˜DIV20 and harvested 10 days post-ASO on ˜DIV30. Summary data for the vehicles is across 49 western blot gels and includes 138 vehicle samples across those gels.

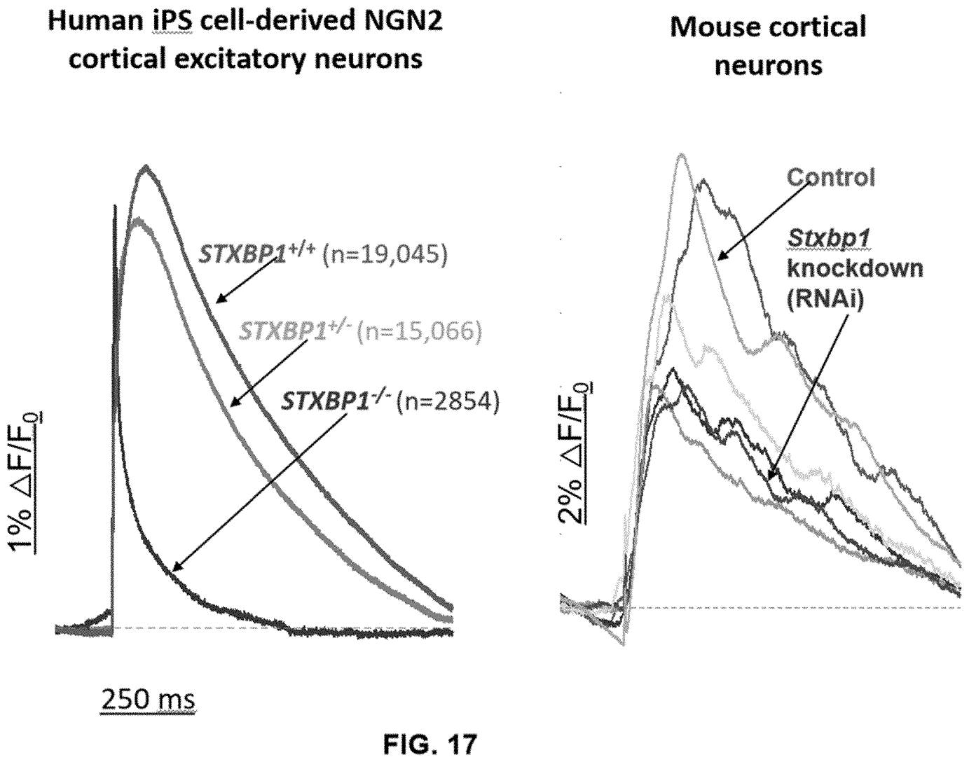

FIGS. 16-17 provide results showing identification of an all-optical electrophysiological synaptic cellular phenotype using the BRITE™ System by Q-State Biosciences, Inc.

Isogenic iPSC-derived neurons were generated using CRISPR editing targeting the STXBP1 gene. CRISPR-edited iPSC clones were selected and screened using Western Blot to genotype neurons from these cell lines. Wild type (WT), heterozygous (Het) knockout, and homozygous knockout (KO) were identified (FIG. 16).

Human iPSC-derived NGN2 excitatory neurons were plated onto 96-well plates for functional optogenetic recordings of evoked synaptic transmission and a dose-dependent STXBP1-associated cellular phenotype was identified in heterozygous (Het) knockout, as shown in FIG. 17: (STXBP1+/−), and homozygous knockout (STXBP1−/−, KO) cell lines as compared to wild type controls (STXBP1+/+). Neurons were measured on day 20. Primary mouse cortical neurons treated with siRNA against STXBP1 shows similar evoked synaptic cellular phenotypes, validating the human CRISPR cell line functional phenotype.

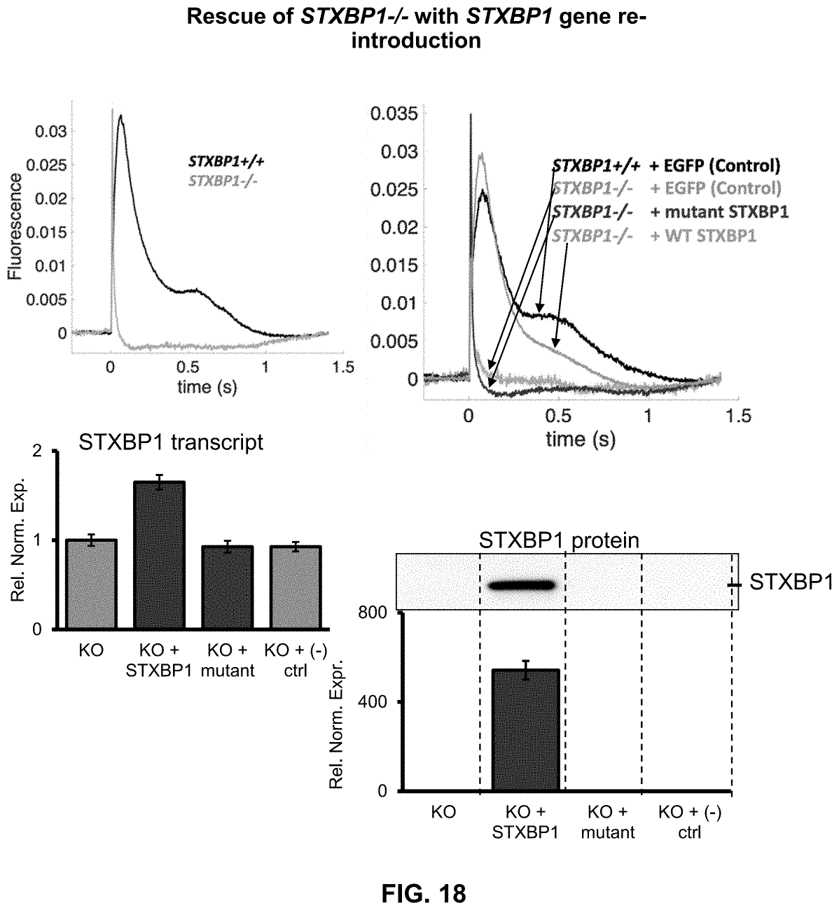

FIG. 18 shows synaptic phenotype rescue by re-introduction of STXBP1 gene via lentiviral delivery. Lentivirus constructs were designed to interrogate the STXBP1 dependence of the synaptic phenotype from FIGS. 16-17. Full length STXBP1, an STXBP1 with a pre-mature stop codon, and a fluorescent tag (EGFP) all under the control of the HSyn promoter were produced for this experiment. STXBP1−/− neurons show a complete loss of synaptic transmission as compared to wild type (STXBP1+/+) neurons (top left), replicating previous data and data presented in FIGS. 16-17. STXBP1−/− neurons were treated with STXBP1 lentivirus, which rescued the deficit in synaptic transmission in these cells. Delivery of a mutant STXBP1 had no effect. Lentivirus was delivered on day 17. Neurons were measured on day 45 and harvested for protein and transcript expression post-imaging. STXBP1 lentivirus-treated KO cells show expression of STXBP1 protein, confirming that the missing protein was successfully re-established in these cells with lentiviral delivery. No protein was noted for non-treated cells or cells treated with EGFP or the mutant STXBP1 (bottom right). Although STXBP1 transcript via qPCR was detected in all conditions, consistent with a lack of nonsense-mediated decay, KO cells treated with the normal STXBP1 lentivirus show upregulation of STXBP1 transcript relative to other conditions.

### Example 2 STXBP1 ASO—3′ Regulatory Region Tiling Program

Based on the positive results for the ASOs developed and tested in Example 1, a tiling approach was conducted to empirically find, and target with ASOs of the invention, 3′ regulatory regions of STXBP1. Using this strategy, ASOs were found that targeted both STXBP1 miRNA sites (SEQ ID NOS: 73-84) or 3′ regulatory regions (SEQ ID NOS: 85-107).

1. A composition comprising:
at least one nucleic acid that promotes expression of Syntaxin binding protein 1 (STXBP1) and has:
at least 50% sequence similarity to one of SEQ ID Nos: 1-3, 7-13, 15-18, and 23-107;
at least 60% sequence similarity to SEQ ID NO: 19;
at least 70% sequence similarity to SEQ ID NO: 22;
at least 75% sequence similarity to SEQ ID NO: 6;
at least 80% sequence similarity to SEQ ID NO: 21;
at least 85% sequence similarity to one of SEQ ID NOS: 14 and 20; or
at least 90% sequence similarity to one of SEQ ID NOS: 4 and 5.

2. The composition of claim 1, wherein the nucleic acid has a length between about 5 and about 50 bases.

3. The composition of claim 1, wherein the nucleic acid has a region of at least about 5 contiguous bases with a 100% match to a segment within one of SEQ ID Nos: 1-25.

4. The composition of claim 1, wherein the nucleic acid comprises at least about 50% RNA
bases with a 2′ modification on a ribose sugar.

5. The composition of claim 1, wherein the at least about 50% of the inter-base linkages in the nucleic acid are not phosphodiester bonds.

6. The composition of claim 1, wherein at least about 12 contiguous bases in the nucleic acid have at least 90% sequence identity to a corresponding about 12 contiguous bases in one of SEQ ID Nos: 1-25.

7. The composition of claim 6, wherein a majority of the bases of the nucleic acid have a 2′-O-methoxyethyl-modified ribose.

8. The composition of claim 7, wherein a majority of inter-base linkages in the nucleic acid are phosphorothioate bonds.

9. The composition of claim 1, wherein all of the bases in the nucleic acid comprise 2′-O-methoxyethyl ribose sugars.

10. The composition of claim 1, wherein the nucleic acid has at least 88% sequence similarity to one of SEQ ID Nos: 1-25 and wherein all of the bases in the nucleic acid comprise 2′-O-methoxyethyl ribose sugars.

11. The composition of claim 1, the nucleic acid has at least 94% sequence similarity to one of SEQ ID Nos: 1-25 and wherein all of the bases in the nucleic acid comprise 2′-O-methoxyethyl ribose sugars.

12. The composition of claim 10 or 11, wherein at least about 90% of inter-base linkages in the nucleic acid are phosphorothioate bonds.

13. The composition of claim 1, wherein: the nucleic acid has 100% sequence similarity to one of SEQ ID Nos: 1-25; all of the bases in the nucleic acid comprise 2′-O-methoxyethyl ribose sugars; all instances of U and Care methylated at position 5; and wherein all inter-base linkages in the nucleic acid are phosphorothioate bonds.

14. The composition of claim 1, further comprising one of the following features (a) through (1):
(a) the nucleic acid hybridizes to a binding site of, and blocks binding of an miR 3p and the nucleic acid has at least 75% sequence similarity to one selecting from the group consisting of SEQ ID Nos: 1, 2, and 3;
(b) the nucleic acid hybridizes to a binding site of, and blocks binding of miR-491-5p and the nucleic acid has at least 90% sequence similarity to one selecting from the group consisting of SEQ ID Nos: 4 and 5;
(c) the nucleic acid hybridizes to a binding site of, and blocks binding of miR-338-3p and the nucleic acid has at least 75% sequence similarity to SEQ ID NO: 6;
(d) the nucleic acid hybridizes to a binding site of, and blocks binding of miR-1-3p and the nucleic acid has at least 75% sequence similarity to one selecting from the group consisting of SEQ ID Nos: 7, 8, 9, 23, 24, and 25;
(e) the nucleic acid hybridizes to a binding site of, and blocks binding of miR-423-5p and the nucleic acid has at least 75% sequence similarity to SEQ ID NO: 10;
(f) the nucleic acid hybridizes to a binding site of, and blocks binding of miR-154-5p and the nucleic acid has at least 75% sequence similarity to one selecting from the group consisting of SEQ ID Nos: 11 and 12;
(g) the nucleic acid hybridizes to a binding site of, and blocks binding of miR-219a-5p and the nucleic acid has at least 75% sequence similarity to SEQ ID NO: 13;
(h) the nucleic acid hybridizes to a binding site of, and blocks binding of miR-424-5p and the nucleic acid has at least 85% sequence similarity to SEQ ID NO: 14;
(i) the nucleic acid hybridizes to a binding site of, and blocks binding of miR-30b-5p and the nucleic acid has at least 75% sequence similarity to one selecting from the group consisting of SEQ ID Nos: 15, 16, and 17;
(j) the nucleic acid hybridizes to a binding site of, and blocks binding of miR-141-3p and the nucleic acid has at least 75% sequence similarity to one selecting from the group consisting of SEQ ID Nos: 18 and 19;
(k) the nucleic acid hybridizes to a binding site of, and blocks binding of miR-218-5p and the nucleic acid has at least 85% sequence similarity to SEQ ID NO: 20 or at least 80% sequence similarity to SEQ ID NO: 21; and
(l) the nucleic acid hybridizes to a binding site of, and blocks binding of miR-143-3p and the nucleic acid has at least 75% sequence similarity to SEQ ID NO: 22.

15. The composition of claim 14, wherein: the nucleic acid has 100% sequence similarity to the one of SEQ ID Nos: 1-25

16-17. (canceled)

18. The composition of claim 1, wherein the nucleic acid has at least 80% sequence identity to one of SEQ ID NOs: 26-49.

19. The composition of claim 18, wherein all of the inter-base linkages are phosphodiester.

20. The composition of claim 1, wherein the nucleic acid has at least 80% sequence identity to one of SEQ ID NOs: 50-73.

21. The composition of claim 20, wherein all of the inter-base linkages are phosphorothioate.

22-29. (canceled)
